SEQUENCE LISTING (11 sequences)

SEQ ID NO: 1 - protein, 449 aa, Homo sapiens
MMKTLLLFVGLLLTWESGQVLGDQTVSDNELQEMSNQGSKYVNKEIQNAVNGVKQIKTLIEKTNEERKTLLSNLEEAKKKKEDALNETRESETKLKELPGVCNETMMALWEECKPCLKQTCMKFYARVCRSGSGLVGRQLEEFLNQSSPFYFWMNGDRIDSLLENDRQQTHMLDVMQDHFSRASSIIDELFQDRFFTREPQDTYHYLPFSLPHRRPHFFFPKSRIVRSLMPFSPYEPLNFHAMFQPFLEMIHEAQQAMDIHFHSPAFQHPPTEFIREGDDDRTVCREIRHNSTGCLRMKDQCDKCREILSVDCSTNNPSQAKLRRELDESLQVAERLTRKYNELLKSYQWKMLNTSSLLEQLNEQFNWVSRLANLTQGEDQYYLRVTTVASHTSDSDVPSGVTEVVVKLFDSDPITVTVPVEVSRKNPKFMETVAEKALQEYRKKHREE

SEQ ID NO: 2 - protein, 8 aa, Homo sapiens
  SITE (2)..(2): Glycan attachment site
HNSTGCLR

SEQ ID NO: 3 - protein, 9 aa, Homo sapiens
  SITE (6)..(6): Glycan attachment site
KEDALNETR

SEQ ID NO: 4 - protein, 10 aa, Homo sapiens
  SITE (7)..(7): Glycan attachment site
KKEDALNETR

SEQ ID NO: 5 - protein, 11 aa, Homo sapiens
  SITE (8)..(8): Glycan attachment site
KKKEDALNETR

SEQ ID NO: 6 - protein, 20 aa, Homo sapiens
  SITE (3)..(3): Glycan attachment site
MLNTSSLLEQLNEQFNWVSR

SEQ ID NO: 7 - protein, 14 aa, Homo sapiens
  SITE (3)..(3): Glycan attachment site
LANLTQGEDQYYLR

SEQ ID NO: 8 - protein, 20 aa, Homo sapiens
  SITE (7)..(7): Glycan attachment site
QLEEFLNQSSPFYFWMWGDR

SEQ ID NO: 9 - protein, 18 aa, Homo sapiens
  SITE (7)..(7): Glycan attachment site
ELPGVCNETMMALWEECK

SEQ ID NO: 10 - protein, 24 aa, Homo sapiens
  SITE (9)..(9): Glycan attachment site
LKELPGVCNETMMALWEECKPCLK

SEQ ID NO: 11 - protein, 8 aa, Homo sapiens
  SITE (5)..(5): Glycan attachment site
EDALNETR

(19) United States
(12) Patent Application Publication
(10) Pub. No.: US 2016/0139151 A1
(43) Pub. Date: May 19, 2016

(54) METHODS AND COMPOSITIONS RELATING TO NEURODEGENERATIVE DISEASES

(71) Applicant: Electrophoretics Limited, Surrey (GB)

(72) Inventors: Malcolm Andrew Ward, Surrey (GB); Hui-Chung Liang, Surrey (GB); Ian Hugo Pike, Surrey (GB)

(73) Assignee: Electrophoretics Limited, Surrey (GB)

(21) Appl. No.: 14/896,018

(22) PCT Filed: Jun. 6, 2014
(86) PCT No.: PCT/GB2014/051758

§ 371 Date: Dec. 4, 2015

(30) Foreign Application Priority Data
Jun. 7, 2013 (GB) 1310150.6

(51) Int. Cl. G01N 33/68

(52) U.S. Cl. CPC G01N 33/6896; G01N 2440/38; G01N 2333/775; G01N 2800/28; G01N 2800/2814; G01N 2800/60; G01N 2458/15; G01N 2800/52

(57) ABSTRACT

The present invention provides a method for diagnosing or assessing a neurodegenerative disease in a test subject, comprising: (i) providing a protein-containing sample that has been obtained from the test subject; (ii) determining the concentration, amount or degree of expression of at least one specific protein isoform and/or glycoform derived from a protein biomarker selected from the group consisting of: clusterin precursor; apolipoprotein A-IV precursor; apolipoprotein C-III precursor; transthyretin; galectin 7; complement C4 precursor; alpha-2-macroglobulin precursor; Ig alpha-1 chain C; histone 2B; Ig lambda chain C region; fibrinogen gamma chain precursor; complement factor H; inter-alpha-trypsin heavy chain H4 precursor; complement C3 precursor; gamma or beta actin; haptoglobin precursor; and the serum albumin precursor, or a fragment thereof; (iii) comparing said concentration, amount or degree determined in (ii) with a reference from a control subject with a specific neurodegenerative disease, dementia or stage of disease, or from a control subject that does not have a neurogenerative disease or dementia; and (iv) based on the level of the at least one specific protein isoform and/or glycoform of the protein biomarker in the test subject relative to the reference, making a diagnosis or assessment as to the presence of and/or stage of neurodegenerative disease or dementia of the test subject. Also provided are related products and systems for use in such a method.

### FIELD OF THE INVENTION

The present invention relates to methods and compositions relating to neurodegenerative diseases, including Alzheimer's disease. Specifically, the present invention identifies and describes protein isoforms that are differentially expressed in the Alzheimer's disease state relative to their expression in the normal state and, in particular, identifies and describes proteins associated with Alzheimer's disease. Further, the present invention provides methods of diagnosis of neurodegenerative diseases, including Alzheimer's disease and other neurodegenerative dementias using the differentially expressed protein isoforms. Still further, the present invention provides methods for the identification and therapeutic use of compounds for the prevention and treatment of neurodegenerative diseases, including Alzheimer's disease and other neurodegenerative dementias.

### BACKGROUND OF THE INVENTION

Dementia is one of the major public health problems of the elderly, and in our ageing populations the increasing numbers of patients with dementia is imposing a major financial burden on health systems around the world. More than half of the patients with dementia have Alzheimer's disease (AD). The prevalence and incidence of AD have been shown to increase exponentially. The prevalence for AD in Europe is 0.3% for ages 60-69 years, 3.2% for ages 70-79 years, and 10.8% for ages 80-89 years (Rocca, Hofman et al. 1991). The survival time after the onset of AD is approximately from 5 to 12 years (Friedland 1993).

Alzheimer's disease (AD), the most common cause of dementia in older individuals, is a debilitating neurodegenerative disease for which there is currently no cure. It destroys neurons in parts of the brain, chiefly the hippocampus, which is a region involved in coding memories. Alzheimer's disease gives rise to an irreversible progressive loss of cognitive functions and of functional autonomy. The earliest signs of AD may be mistaken for simple forgetfulness, but in those who are eventually diagnosed with the disease, these initial signs inexorably progress to more severe symptoms of mental deterioration. While the time it takes for AD to develop will vary from person to person, advanced signs include severe memory impairment, confusion, language disturbances, personality and behaviour changes, and impaired judgement. Persons with AD may become non-communicative and hostile. As the disease ends its course in profound dementia, patients are unable to care for themselves and often require institutionalisation or professional care in the home setting. While some patients may live for years after being diagnosed with AD, the average life expectancy after diagnosis is eight years.

In the past, AD could only be definitively diagnosed by brain biopsy or upon autopsy after a patient died. These methods, which demonstrate the presence of the characteristic plaque and tangle lesions in the brain, are still considered the gold standard for the pathological diagnoses of AD. However, in the clinical setting brain biopsy is rarely performed and diagnosis depends on a battery of neurological, psychometric and biochemical tests, including the measurement of biochemical markers such as the ApoE and tau proteins or the beta-amyloid peptide in cerebrospinal fluid and blood.

Biomarkers may possibly possess the key in the next step for diagnosing AD and other dementias. A biological marker that fulfils the requirements for the diagnostic test for AD would have several advantages. An ideal biological marker would be one that identifies AD cases at a very early stage of the disease, before there is degeneration observed in the brain imaging and neuropathological tests. A biomarker could be the first indicator for starting treatment as early as possible, and also very valuable in screening the effectiveness of new therapies, particularly those that are focussed on preventing the development of neuropathological changes. Repetitive measurement of the biological markers of the invention would also be useful in following the development and progression of the disease.

Markers related to pathological characteristics of AD; plaques and tangles (Aβ and tau) have been the most extensively studied. The most promising has been from studies of CSF concentration of Aβ(1-40), Aβ(1-42) and tau or the combination of both proteins in AD. Many studies have reported a decrease in Aβ(1-42) in CSF, while the total Aβ protein or Aβ(1-40) concentration remain unchanged (Ida, Hartmann et al. 1996; Kanai, Matsubara et al. 1998; Andreasen, Hesse et al. 1999).

Recognising that CSF is a less desirable sample and that ‘classical’ markers of AD pathology including amyloid and tau are not reliably detectable in other fluids, there have been several efforts to identify additional protein markers in blood and blood products such as serum and plasma. One group of blood proteins that are differentially expressed in the AD state relative to their expression in the normal state are described in WO2006/035237 and includes the protein clusterin which has previously been associated with AD pathology in the brain of affected individuals. The value of clusterin as a potential biomarker in AD has been explored by various groups in both cerebrospinal fluid (CSF) and blood, often with contradictory results. One possible explanation for the discrepancy between CSF clusterin levels and those found in the brain is the effect of protein glycosylation which may serve to mask epitopes recognised by antibodies used in immunoassays to measure clusterin. Indeed, Nilselid et al. (2006) demonstrated that accurate quantification of clusterin in human CSF was only possible when all glycan moieties were enzymatically removed from clusterin prior to measurement by ELISA. In their study, they found that the clusterin amount measured by two specific antibodies to the alpha and beta chains of clusterin increased by approximately 70% following deglycosylation. Importantly, although clusterin levels were generally elevated in male AD patients relative to healthy male controls their study failed to show diagnostic utility for measuring CSF levels of either the naturally glycosylated clusterin levels, or those of the ex vivo deglycosylated protein. Furthermore, they found no difference in clusterin levels between women with AD and the female control group. The authors conclude that there was no general difference in clusterin glycosylation levels between AD and control groups but rather contradict this by suggesting that protein microheterogeneity (glycosylation, phosphorylation etc) could be another useful target in the diagnosis or prognostic monitoring of disease.

### SUMMARY OF THE INVENTION

In light of this uncertain art and wishing to develop a minimally invasive diagnostic test using blood rather than CSF, the inventors have surprisingly shown that glycosylation of clusterin in human plasma is highly heterogenous with over 40 different isoforms identified to date. Furthermore, a small subset of only 8 of the identified glycoforms is consistently regulated between patients with AD and those with Mild Cognitive Impairment. Furthermore, levels of these same glycoforms can predict the severity and rate of progression of AD within an individual.

The present inventors have previously determined a number of plasma biomarkers for Alzheimer's disease (see U.S. Pat. No. 7,897,361; the contents of which are incorporated herein by reference). However, they found that immunoassays and selected reaction monitoring experiments did not fully replicate the results they obtained for the same biomarkers using 2-dimensional gel electrophoresis (2DE). The inventors investigated whether this difference could be due to specific post-translational events which were not being replicated in the validation experiments.

The inventors surprisingly found that post-translational events created distinct isoforms of the protein, e.g. glycoforms, which were differentially expressed in different forms and stages of dementia. Accordingly, the inventors have identified more potent biomarkers for dementia and as a result can provide more sophisticated methods for the diagnosis, prognosis and monitoring of dementia such as Alzheimer's disease.

In particular, the inventors provide herein examples of blood proteins useful in the diagnosis and prognostic monitoring of AD and other forms of dementia that carries extensive post-translational modifications (PTMs)—and wherein measurement of total protein level lacks sufficient diagnostic power whereas measurement of specific isoforms allows accurate diagnosis and prognostic assessment of disease.

Broadly, the present invention relates to methods and compositions for the diagnosis of neurodegenerative diseases, including dementia, specifically Mild Cognitive Impairment (MCI) (a recognised precursor to AD), AD and other late onset dementias including vascular dementia, dementia with lewy bodies and frontotemporal dementia, alone and as a mixed dementia with Alzheimer's disease.

The present inventors have identified and described proteins each having one or more isoforms that are differentially expressed in the MCI and AD states relative to each other and/or their expression in the normal state.

A protein in vivo can be present in several different forms. These different forms may be produced by alternative splicing; by alterations between alleles, e.g. single nucleotide polymorphisms (SNPs); or may be the result of post translational events such as glycosylation (glycoforms). A glycoform is an isoform of a protein that differs only with respect to the number or type of attached glycan.

The invention relates to the determination of one or more different isoforms (preferably glycoforms) of a particular protein where said one or more isoforms are present to a greater or lesser extent in subjects with a neurodegenerative disease or dementia (e.g. MCI or AD) than in healthy (e.g. non-dementia) subjects. Determining the level of the one or more isoforms in a subject (with or without comparison to a reference level) allows the skilled practitioner to diagnose the neurodegenerative disease or dementia and/or the level, nature and extent of said neurodegenerative disease or dementia.

In all aspects of the present invention, the isoforms are derived from protein biomarkers selected from the group consisting of clusterin precursor, apolipoprotein A-IV precursor, apolipoprotein C-III precursor, transthyretin, galectin 7, complement C4 precursor, alpha-2-macroglobulin precursor, Ig alpha-1 chain C, histone 2B, Ig lambda chain C region, fibrinogen gamma chain precursor, complement factor H, inter-alpha-trypsin heavy chain H4 precursor, complement C3 precursor, gamma or beta actin, haptoglobin precursor or the serum albumin precursor isoform.

In preferred embodiments, the protein biomarker is selected from the group consisting of alpha-2-macroglobulin precursor, fibrinogen gamma chain precursor, complement factor H, clusterin and haptoglobin.

In a further preferred embodiment, the protein biomarker is clusterin (e.g. human, mouse or rat clusterin, particularly human clusterin having the amino acid sequence disclosed at UNIPROT Accession Number P10909; SEQ ID NO: 1).

It will be understood that any one or more of these biomarkers may be used in the methods of the invention. For example, several biomarkers may be selected to create a biomarker panel comprising a plurality of biomarkers, e.g. at least clusterin and optionally alpha-2-macroglobulin precursor, fibrinogen gamma chain precursor, complement factor H, and haptoglobin.

Although the invention concerns the detection and quantification of isoforms from proteins which demonstrate differential abundance in dementia subjects compared to normal subjects, the inventors arrived at the invention through their work on clusterin. However, it will be apparent to the skilled practitioner that the examples provided herein will allow the invention to be carried out using other glycosylated protein biomarkers.

In all aspects, the methods of the present invention may be used in relation to all forms of neurodegenerative disease or dementia, but particularly to pre-Alzheimer's stages such as mild cognitive impairment (MCI) as well as advanced Alzheimer's disease. For convenience however, the following aspects and embodiments of the invention refer to MCI and AD specifically. However, it is to be understood that the methods may equally relate to neurodegenerative disease or dementia in general or to specific forms of dementia other than MCI and AD, alone or in combination.

In a first aspect, the invention provides a method of diagnosing or assessing a neurodegenerative disease or neurodegenerative dementia, such as Alzheimer's disease, in a subject, the method comprising detecting one or more different isoforms, preferably glycoforms, of a protein biomarker in a tissue sample or body fluid sample from said subject.

Preferably, the method is an in vitro method (e.g. carried out on a sample that has been isolated, extracted or otherwise obtained from the subject).

Preferably the protein biomarker selected from the group consisting of apolipoprotein A-IV precursor, apolipoprotein C-III precursor, transthyretin, galectin 7, complement C4 precursor, alpha-2-macroglobulin precursor, Ig alpha-1 chain C, histone 2B, Ig lambda chain C region, fibrinogen gamma chain precursor, complement factor H, inter-alpha-trypsin heavy chain H4 precursor, complement C3 precursor, clusterin precursor, gamma or beta actin, haptoglobin precursor or the serum albumin precursor isoform.

In preferred embodiments, the protein biomarker is selected from the group consisting of alpha-2-macroglobulin precursor, fibrinogen gamma chain precursor, complement factor H, clusterin and haptoglobin.

In a further preferred embodiment, the protein biomarker is clusterin (e.g. human clusterin having the amino acid sequence disclosed at UNIPROT Accession Number P10909; SEQ ID NO: 1). It will be understood by the skilled person that the equivalent clusterin sequences from species other than human (e.g. other mammalian species, such as non-human primates, rodents (e.g. mouse or rat), laboratory animals and the like) may be substituted in the present invention. For example, when using the invention to determine efficacy of new treatments for neurodegenerative dementia in a rodent model of disease the appropriate rodent species sequence should be used (e.g. mouse clusterin (UniProt accession number Q06890, sequence version 1, dated 1 Feb. 1995) or rat clusterin (UniProt accession number P05371, sequence version 2, dated 1 Feb. 1994)).

For each of the biomarkers listed above, the invention provides one or more isoforms in a biomarker panel which may be used in combination to establish an isoform profile for the subject. This profile may be compared with reference profiles, profiles taken previously from the same subject or profiles taken from a control subject.

For all aspects, the biomarker panel may comprise two or more, three or more, four or more, or five or more isoforms.

For all aspects, a plurality of biomarker panels may be used, each relating to a different protein marker protein, e.g. clusterin and alpha-2-macroglobulin precursor.

In accordance with the present invention there is provided a method for diagnosing or assessing a neurodegenerative disease or neurodegenerative dementia in a test subject, comprising:
(i) providing a protein-containing sample that has been obtained from the test subject;
(ii) determining the concentration, amount or degree of expression of at least one specific protein isoform and/or glycoform derived from a protein biomarker selected from the group consisting of: clusterin precursor; apolipoprotein A-IV precursor; apolipoprotein C-III precursor; transthyretin; galectin 7; complement C4 precursor; alpha-2-macroglobulin precursor; Ig alpha-1 chain C; histone 2B; Ig lambda chain C region; fibrinogen gamma chain precursor; complement factor H; inter-alpha-trypsin heavy chain H4 precursor; complement C3 precursor; gamma or beta actin; haptoglobin precursor; and the serum albumin precursor, or a fragment thereof;
(iii) comparing said concentration, amount or degree determined in (ii) with a reference from a control subject with a specific neurodegenerative disease, dementia or stage of disease, or a control subject that does not have a neurodegenerative disease or does not have a neurodegenerative dementia; and
(iv) based on the level of the at least one specific protein isoform and/or glycoform of the protein biomarker in the test subject relative to the reference, making a diagnosis or assessment as to the presence of and/or stage of neurodegenerative disease or neurodegenerative dementia of the test subject.

In some cases the at least one specific protein isoform and/or glycoform is derived from clusterin precursor. In particular, said at least one specific protein isoform and/or glycoform may comprise:
a glycoform of human clusterin; or
a glycosylated fragment of human clusterin comprising at least 5, 6, 7, 8, 9, or at least 10 contiguous amino acids of the human clusterin amino acid sequence, wherein said fragment comprises an N-linked or O-linked glycan. Particular glycosylated fragments of human clusterin contemplated for use in accordance with the present invention include:

(SEQ ID NO: 2)
HN*STGCLR;
(SEQ ID NO: 3)
KEDALN*ETR;
(SEQ ID NO: 4)
KKEDALN*ETR;
(SEQ ID NO: 5)
KKKEDALN*ETR;
(SEQ ID NO: 6)
MLN*TSSLLEQLNEQFNWVSR;
(SEQ ID NO: 7)
LAN*LTQGEDQYYLR;
and
(SEQ ID NO: 8)
QLEEFLN*QSSPFYFWMWGDR;
(SEQ ID NO: 9)
ELPGVCN*ETMMALWEECK;
(SEQ ID NO: 10)
LKELPGVCN*ETMMALWEECKPCLK,
wherein “N*” indicates the glycan attachment
residue.

In some cases in accordance with the present invention said glycosylated fragment of human clusterin is selected from any one of the clusterin glycopeptides set forth in Table 3A, Table 3B, Table 3C, Table 5, Table 6 and/or Table 7.

In some cases in accordance with the present invention said glycosylated fragment of human clusterin comprises a β64N-glycan selected from the group consisting of: β64N_SA1-(HexNAc-Hex)2-core; β64N_SA2-(HexNAc-Hex)2-core; β64N_SA1-(HexNAc-Hex)3-core; β64N_SA2-(HexNAc-Hex)3-core; β64N_SA1-(HexNAc-Hex)4-core; β64N_SA3-(HexNAc-Hex)3-core; β64N_SA2-(HexNAc-Hex)4-core; and β64N_SA3-(HexNAc-Hex)4-core.

In some cases in accordance with the present invention the at least one specific protein glycoform is a tetra-antennary glycoform of the protein biomarker.

In some cases in accordance with the present invention the concentration, amount or degree of expression of the at least one specific protein isoform and/or glycoform is determined
(i) relative to at least one other isoform and/or glycoform of the same protein or relative to the total of all isoforms and/or glycoforms of the same protein;
(ii) relative to a reference protein other than one of said protein biomarkers; or
(iii) using a sum-scaling method in which one or more raw values of said concentration, amount or degree of expression are normalised to give a normalised sum-scaled measurement. In particular, the concentration, amount or degree of expression of a tetra-antennary glycoform of the protein biomarker may be determined relative to one or more lower antennary glycoforms (e.g. tri-antennary or bi-antennary glycoforms) of the same protein or relative to the total of all glycoforms of the same protein.

In certain cases the method of the present invention comprises determining the proportion of tetra-antennary glycoforms of the protein biomarker relative to the total of all glycoforms of the same protein.

In certain cases the method of the present invention comprises quantifying tetra-antennary glycoforms of the human clusterin glycoprotein fragment comprising or consisting of the sequence HN*STGCLR (SEQ ID NO: 2) as a proportion of the total of all glycoforms of the same glycoprotein fragment.

In certain cases of the method of the present invention a lower relative level of tetra-antennary glycoforms in the sample from the test subject compared with the relative level of tetra-antennary glycoforms in the reference from the control subject indicates that the test subject has or is predicted to have a neurodegenerative disease or dementia and/or to have a more advanced stage of neurodegenerative disease or dementia. In particular, this may indicate that the subject has a relatively higher level of hippocampal atrophy.

In accordance with the present invention, the neurodegenerative disease or neurodegenerative dementia may be selected from the group consisting of: Alzheimer's disease (AD), Mild Cognitive Impairment (MCI), vascular dementia, dementia with Lewy bodies, frontotemporal dementia alone or as a mixed dementia with AD, Parkinson's disease, and Huntington's disease.

In certain cases the method of the present invention comprises determining the concentration, amount or degree of expression of at least one specific protein isoform and/or glycoform of each of at least two, three, four or at least five of said biomarker proteins.

In certain cases the method of the present invention comprises determining the concentration, amount or degree of expression of at least two, three, four or at least five specific protein isoforms and/or glycoforms of the, or of each of the, protein biomarkers.

In certain cases in accordance with the present invention, the protein-containing sample is selected from the group consisting of: blood plasma, blood cells, serum, saliva, urine, cerebro-spinal fluid (CSF), cell scraping, and a tissue biopsy.

The skilled person will be aware that a variety of suitable techniques exist for measuring the amount or concentration of specific protein isoforms, including specific glycoforms. This includes the use of non-human antibodies generated by immunisation with specific isoforms of the proteins if the present invention wherein such antibodies have the required specificity for the diagnostic isoform, particularly glycoforms. In particular, the use of synthetic peptides of Sequence ID's 2-10 with the appropriate glycan structures. Such peptides are not found in nature and must therefore be prepared ex vivo through digestion of naturally occurring clusterin or by the use of in vitro synthetic chemistry.

More specifically contemplated herein are methods that include measurement using gel electrophoresis or LC-MS/MS.

In some cases the relative amount of each glycoform is calculated by comparison to an equivalent heavy-isotope labelled reference glycoform using Selected Reaction Monitoring mass spectrometry. In particular, the heavy-isotope labelled reference glycoform may be a synthetic glycopeptide in which one or more heavy isotopes of H, C, N or O are substituted within the peptide or sugar components of said glycoform.

In some cases the heavy-isotope labelled reference glycoform is an enriched, naturally occurring glycoform that has been labelled with an isotopic mass tag wherein said isotopic mass tag with one or more heavy isotopes of H, C, N or O and wherein such mass tag is able to react with the peptide or sugar components of said glycoform.

In some cases the relative amount of each glycoform is calculated by comparison to an equivalent glycoform labelled with an isobaric mass tag as generally disclosed in European Patent 2,115,475 (the entire content of which is incorporated herein by reference) wherein:
(i) each sample of tissue or body fluid taken from the test subject is labelled with one member of an isobaric mass tag set to create a labelled analytical sample;
(ii) a standard reference panel of enriched glycoforms is separated into between two and six aliquots and each aliquot is labelled separately with additional members of the same isobaric mass tag set as the labelled analytical sample and each independently labelled aliquot of the reference panel is mixed in a predefined ratio to create a clinically relevant concentration curve as a standard reference mixture;
(iii) an equal volume of the labelled analytical sample and the standard reference mixture are mixed together to form the MScalibrator sample; and
(iv) the MScalibrator sample prepared in step (iii) is analysed by mass spectrometry. In particular, the the isobaric mass tag set may be a Tandem Mass Tag set.

In certain cases in accordance with the present invention, the protein-containing sample is selected from the group consisting of: blood plasma, blood cells, serum, saliva, urine, cerebro-spinal fluid (CSF), cell scraping, and a tissue biopsy.

In certain cases in accordance with the present invention, the protein isoforms and/or glycoforms are glycoforms and are measured using sum scaled Selected Reaction Monitoring (SRM) mass spectrometry.

In certain cases in accordance with the present invention, the protein isoforms and/or glycoforms are not labelled.

In certain cases in accordance with the present invention, the method does not comprise subjecting the sample to gel electrophoretic separation, and/or does not comprise subjecting the sample to enrichment by immunoprecipitation.

In certain cases in accordance with the present invention, the protein isoforms and/or glycoforms are glycoforms and are measured by a method essentially as described in Example 6.

In some cases in accordance with the present invention the at least one specific protein isoform and/or glycoform may be measured by an immunological assay, such as Western blotting or ELISA.

In some cases in accordance with the present invention the method comprises determining the relative profile of at least 5, 6, 7, 8, 9 or at least 10 glycopeptides as set forth in Table 1A or 1B herein. In particular, the relative percentages of said glycopeptides in the sample from the test subject may be compared with the relative percentages of said glycopeptides as set forth in column “AVG_A” and/or “AVG_B” in Table 1A.

In some cases in accordance with the present invention the method comprises identifying said glycopeptides at least in part by reference to the retention time, m/z value and/or charge state values set forth in Table 1A or 1B.

In a further aspect the present invention provides a method for stratifying a plurality of test subjects according to their stage and/or severity of neurodegenerative disease or dementia, comprising:
carrying out the method according to the first aspect of the invention on at least one test sample from each of the test subjects; and
based on the level of the at least one specific protein isoform and/or glycoform of the protein biomarker in each of the test subjects, stratifying the test subjects into more or less advanced stage neurodegenerative disease or dementia or into more or less severe neurodegenerative disease or dementia. In particular, the test subjects may be stratified according to their predicted degree of hippocampal atrophy.

Accordingly, the present invention provides a method of diagnosing or assessing a neurodegenerative condition in a subject comprising the steps of;
(i) obtaining a sample of a relevant tissue or body fluid from a test subject suspected of having or previously diagnosed with dementia wherein such sample comprises one or more protein isoforms of a biomarker; and
(ii) detecting one or more protein isoforms in a biomarker panel for said biomarker in said relevant tissue sample or body fluid; and
(iii) comparing the presence or amount of the said one or more protein isoforms to the levels of the said protein isoforms in a representative sample of the equivalent relevant tissue or body fluid sample taken either from a control subject with a specific dementia or stage of disease, or a control subject that does not have dementia; and
(iv) based on the relative level of the one or more isoforms in the test subject relative to the control subject making a diagnosis as to the presence and/or stage of dementia.

Preferably, the biomarker panel comprises one or more glycoforms of a biomarker.

The detection of the isoforms, preferably glycoforms, may be carried out by using gel electrophoresis, but more preferably by LC-MS/MS.

In another aspect, the present invention provides a method of determining the nature or degree of dementia, e.g. MCI or AD, in a human or animal subject, the method comprising detecting one or more isoforms of a protein biomarker in a tissue sample or body fluid sample from said subject. Thus, the methods of the present invention encompass methods of monitoring the progress of Alzheimer's disease or of disease progression from MCI to Alzheimer's disease. Also encompassed are prognostic methods, for example prognosis of likely progression from MCI to Alzheimer's disease, or prognosis of likely duration or severity of Alzheimer's disease.

Preferably the protein biomarker is selected from the group consisting of apolipoprotein A-IV precursor, apolipoprotein C-III precursor, transthyretin, galectin 7, complement C4 precursor, alpha-2-macroglobulin precursor, Ig alpha-1 chain C, histone 2B, Ig lambda chain C region, fibrinogen gamma chain precursor, complement factor H, inter-alpha-trypsin heavy chain H4 precursor, complement C3 precursor, clusterin precursor, gamma or beta actin, haptoglobin precursor or the serum albumin precursor isoform.

In preferred embodiments, the protein biomarker is selected from the group consisting of alpha-2-macroglobulin precursor, fibrinogen gamma chain precursor, complement factor H, clusterin and haptoglobin.

In a further preferred embodiment, the protein biomarker is clusterin (UNIPROT Accession Number P10909; (SEQ ID NO: 1).

In a preferred aspect of the invention there is provided a method comprising:
(a) obtaining a sample of the tissue or body fluid sample from the subject;
(b) determining the concentration, presence, absence or degree of one or more isoforms of a biomarker or of biomarkers in the sample; and
(c) relating the determination to the nature or degree of dementia by reference to a previous correlation between such a determination and clinical information; or by reference to a determination made on a non-dementia subject.

In a preferred embodiment, the progression of dementia (e.g. MCI to AD) may be determined by sequential determinations over a period of time and comparisons made between the concentration, presence, absence or degree of the one or more isoforms of a biomarker over different time points.

The determination may be related to the nature or degree of the AD in the subject by reference to a previous correlation between such a determination and clinical information in control patients. Alternatively the determination of progression or severity may be made by comparison to the concentration, amount or degree of expression of the said protein isoforms in an earlier sample taken from the same subject. Such earlier sample may be taken one week, one month, three months and more preferably six months before the date of the present test. It is also a feature of the present invention that multiple such earlier samples are compared in a longitudinal manner and the slope of change in protein isoform expression is calculated as a correlate of cognitive decline.

Preferably the biomarker is selected from the group consisting of apolipoprotein A-IV precursor, apolipoprotein C-III precursor, transthyretin, galectin 7, complement C4 precursor, alpha-2-macroglobulin precursor, Ig alpha-1 chain C, histone 2B, Ig lambda chain C region, fibrinogen gamma chain precursor, complement factor H, inter-alpha-trypsin heavy chain H4 precursor, complement C3 precursor, clusterin precursor, gamma or beta actin, haptoglobin precursor or the serum albumin precursor isoform.

In preferred embodiments, the biomarker is selected from the group consisting of alpha-2-macroglobulin precursor, fibrinogen gamma chain precursor, complement factor H, clusterin and haptoglobin.

In a further preferred embodiment, the biomarker is clusterin (UNIPROT Accession Number P10909; (SEQ ID NO: 1).

It is a further aspect of the invention that the determined level of the protein isoforms of the biomarker panel are used in conjunction with other clinical and laboratory assessments to increase the level of confidence of a diagnosis of MCI, AD, and other late onset dementias including vascular dementia, dementia with lewy bodies and frontotemporal dementia, alone and as a mixed dementia with Alzheimer's disease.

In one embodiment, the progression of the disorder may be tracked by using the methods of the invention to determine the severity of the disorder, e.g. global dementia severity. In another embodiment, the duration of the disorder up to the point of assessment may be determined using the methods of the invention.

This method allows the type of dementia, e.g. Alzheimer's disease, of a patient to be correlated to different types to prophylactic or therapeutic treatment available in the art, thereby enhancing the likely response of the patient to the therapy.

In some embodiments, one or more, two or more, or three or more different isoforms of a particular protein are detected and quantified in a sample in order to carry out the method of the invention. In a further preferred embodiment, the isoforms of more than one protein are detected, thereby providing a multi-protein fingerprint of the nature or degree of the Alzheimer's disease. Preferably, the one or more isoforms of at least four different proteins detected.

Conveniently, the patient sample used in the methods of the invention can be a tissue sample or body fluid sample such as urine, blood, plasma, serum, salvia or cerebro-spinal fluid sample. Preferably the body fluid sample is blood, serum or plasma sample. Use of body fluids such as those listed is preferred because they can be more readily obtained from a subject. This has clear advantages in terms of cost, ease, speed and subject wellbeing. Blood, blood products such as plasma or serum and urine are also particularly preferred.

The step of detecting the protein isoforms of the specified one or more proteins may be preceded by a depletion step to remove the most abundant proteins from the sample or by targeted enrichment of the proteins included in the biomarker panel, in each case using methods that are well known in the art, e.g. such as immune capture or one- or two-dimensional gel electrophoresis.

Any of the protein isoforms as described herein may be differentially expressed (i.e. display increased or reduced expression) or uniquely present or absent in normal samples or tissue relative to samples or tissue from a subject with dementia e.g. MCI or AD. It should be understood by the skilled practitioner that it is not required that all the protein isoforms of the protein are differentially expressed within the individual subject and that the number and identity of the differentially expressed protein isoforms seen in any individual test will vary between different subjects and for an individual subject over time. Specific subsets of the protein isoforms may be used for different purposes such as diagnosis, prognosis and estimation of disease duration. For each protein a minimum number of differentially expressed protein isoforms is required to provide a secure determination. In preferred embodiments a minimum of one protein isoform, more preferably at least two and most preferably three or more protein isoforms are differentially expressed. The said one, two, three or more isoforms may all be isoforms of a single protein or may be isoforms of more than one protein.

Preferably, at least one of the differentially expressed protein isoforms is an isoform of the glycoprotein clusterin (UNIPROT Accession Number P10909; (SEQ ID NO: 1) which is processed after expression into two distinct alpha and beta chains which associate to form heterodimers, or proteolytic fragments thereof wherein said clusterin protein or proteolytic fragment comprises at least one N-linked or O-linked glycan structure.

It is most preferred that the one or more isoforms detected in accordance with the invention comprise differentially glycosylated isoforms of human clusterin. In particular the inventors have unexpectedly found that truncation and/or complete removal of glycan antennary components occur differentially in MCI, AD and other dementias. It is also a feature of the present invention that specific antennary forms of N-linked glycans on clusterin are associated with the level of hippocampal atrophy, a well-known marker of disease severity in AD and MCI.

Methods for detecting the one or more protein isoforms of a selected protein are well known in the art and may include mass spectrometry, immune-mass spectrometry, immunoassays such as Western blotting or ELISA, lectin affinity immunoassays, gel electrophoresis, 2-dimensional gel electrophoresis and iso-electric focusing.

Accordingly, the measurement of glycan structures on clusterin may be performed by various methods. In 2-dimensional gel electrophoresis the addition or removal of sugar groups within the glycan structure will affect both the apparent molecular mass and the iso-electric focusing point of clusterin leading to a ‘train’ of spots within the gel. Such trains of spots are well known to the skilled practitioner. By way of example, a plasma protein from a subject suspected of suffering from dementia is subjected to 2-dimensional gel electrophoresis. After completion of the second dimension the gel is stained with a protein or sugar-selective dye to reveal individual protein spots or glycoprotein spots respectively. Typically an image of the whole gel is captured using a CCD camera and the relative abundance of each spot calculated based on staining intensity using commercially available software such as SameSpots (Non-Linear Dynamics, UK). The train of spots comprising clusterin isoforms can be identified by comparison with a reference gel. Alternatively, spots can be cut from the gel and proteins identified using mass spectrometry. Ultimately, the relative abundance of each spot representing the different clusterin isoforms is determined and the level of the diagnostic and/or prognostic isoforms compared to those known to represent AD, MCI or other dementias.

Accordingly, the invention provides a method of diagnosing dementia, particularly Alzheimer's disease, in a subject, the method comprising detecting an isoform of clusterin (Swiss-PROT Accession number (SPN) P10909; (SEQ ID NO: 1) in a body fluid sample obtained from said subject, wherein a change in the relative abundance of said isoform is indicative in dementia in said subject. The relative abundance of said isoform may be determined by comparing the detected concentration or abundance with the concentration or abundance of the same isoform in a previous sample from the same subject taken at least one month, at least two months, at least three months, at least 6 months, at least one year, at least two years or at least five years previously, or by comparing the detected concentration or abundance with the concentration or abundance of the same isoform from reference samples (said reference samples may conveniently form a database); or by comparing the detected concentration or abundance with the concentration or abundance of the same isoform from a sample obtained from a non-dementia control subject.

Preferably, in respect of clusterin, the one or more isoforms are selected from Table 1A or 1B. More preferably, two or more, three or more, four or more, five or more, 10 or more, or 20 or more isoforms are selected from Table 1A or 1B. In a further preferred embodiment, the one or more isoforms of clusterin are sialylated forms of glycopeptide HN*STGCLR (SEQ ID NO: 2).

In a further embodiment, the invention provides a method for detecting specific N-linked and/or O-linked glycan structures of clusterin by liquid chromatography tandem mass spectrometry (LC-MS/MS). Optionally, clusterin protein of all isoforms is enriched from a biological tissue or fluid sample, e.g. a plasma sample, using an antibody recognising a region of the unmodified protein backbone in a method such as immunoprecitipitation or immunoaffinity chromatography.

Such clusterin-specific antibodies are well known in the art. Alternatively lectin affinity precipitation or lectin affinity chromatography may be used to perform enrichment of specific glycoforms, typically using lectins such as wheat germ agglutinin. Following enrichment the naturally occurring clusterin is transformed by subjecting the enriched protein fraction to proteolytic digestion using an enzyme such as Trypsin or Asp-N prior to separation of the peptide fragments by reverse-phase liquid chromatography linked to a mass spectrometer. During the mass spectrometry analysis the abundance of each clusterin peptide is determined in the MS1 survey scan. Each peptide is then subjected to fragmentation within the mass spectrometer to break the peptide backbone and release attached glycans. In each case the exact mass of the released fragments is determined in the MS2 scan and can be used to identify the peptide sequence and glycan structure. Thus a relative quantitation of each clusterin isoform is obtained and can be compared to the known amounts of each isoform associated with a particular form of dementia, stage of disease progression or non-demented control.

In an even more preferred embodiment a reference panel of isotopically or isobarically labelled glycoprotein(s) and/or glycopeptides representing the protein isoforms are added to the sample of tissue or body fluid taken from a subject suspected of having, or previously diagnosed with dementia prior to subsequent analysis by LC-MS/MS.

In one such aspect the specific glycopeptides are quantified using a TMT-SRM approach (as disclosed in Byers et al., J. Proteomics 73: 231-239 the entire content of which is incorporated herein by reference) whereby the endogenous amount of the analyte is measured against a reference panel comprising an enriched preparation of the different isoforms of clusterin prepared from a universal donor sample, e.g. a plasma sample, and labelled with a heavy TMT reagent. The ‘heavy’ reference is added into a similarly prepared enriched endogenous clusterin prepared from the sample of tissue or bodily fluid taken from a subject suspected of having, or previously diagnosed with dementia which is labelled with a light TMT reagent.

This mixture of heavy reference and light endogenous clusterin is then subjected to LC-MS/MS and the relative abundance of the equivalent heavy and light parent and daughter ions (so called SRM transitions) each representing the sequential loss of glycan units from successive fragment ions observed in MS/MS experiments is calculated. Where appropriate, transitions measuring m/z 366.14 and m/z 657.24 would also be included. These ions relate to hexose-N-acetylhexosamine, [Hex-HexNAc]⁺, and N-acetylneuraminic acid-hexose-N-acetylhexosamine [NeuAc-Hex-HexNAc]⁺ respectively and are typically created during collision induced dissociation of glycopeptides containing N-linked carbohydrates. The ratio of light TMT/heavy TMT for each SRM transition is thus directly proportional to the relative abundance of the relevant glycopeptide. The measured level is then compared against the known reference levels for the relevant isoform found in the appropriate tissue or bodily fluid taken from subjects with AD, MCI or other dementias and/or non-demented control subjects to enable diagnosis and/or prediction of disease state or rate of progression.

It is particularly preferred that the reference panel comprises isobarically labelled glycopeptides and that two or more different concentrations of each glycopeptide are included in the reference panel. Any isobaric protein or sugar tag such as Tandem Mass Tags (Thermo Scientific, UK) may be used. The principles of this so called TMTcalibrator method are disclosed in European Patent 2115475 the subject matter of which is fully incorporated herein.

In an alternative embodiment the invention provides for the use of Selected Reaction Monitoring of the key glycoform peptides of clusterin where quantification is provided by an unrelated reference peptide. In this method a peptide that provides a strong SRM signal and does not interfere with the clusterin glycoform peptide ionisation and detection may be added to each patient sample after preparation of the clusterin glyopeptides. This mixture is then subjected to the SRM method and the relative peak area of the clusterin glycoform peptide transitions is compared to that of the reference peptide to give a relative or absolute quantification.

In another SRM method embodied by the invention there is no reference peptide added to the mixture. In such a method the values of raw integrated peak area of each glycosylated peptide (analyte) are used for quantification, but first normalised using sum-scaling. Sum scaling is a mathematical approach to remove experimental bias (see Robinson et al., 2010; Paulson et al., 2013; and De Livera et al., 2012). The process involves summing the intensity values for all analytes measured in a given sample and then calculating the median value across all the samples. The median value is then divided by each summed value to create a correction factor which is then multiplied to the original intensity values to give the normalised sum scaled measurement.

The median values were calculated between high and low atrophy. Homoscedastic one tailed distribution t-test was used to calculate p-values. In addition, log 2 ratios were also calculated to provide the regulation between high atrophy over low atrophy for each glycosylated peptide. A glycopeptide high atrophy/low atrophy log 2 ratio is the median value of high atrophy/low atrophy log 2.

In a further aspect, the invention provides a database of glycopeptides retention time, precursor mass and diagnostic fragmentation masses for all protein isoforms of the marker protein panel. An example of such a database is provided in Table 1A or 1B. Preferably the database also comprises a spectral library of high mass accuracy MS and MS/MS spectra collected on FTMS and/or QTOF instruments.

In a further aspect the present invention provides a method of determining the efficacy of a treatment of a neurodegenerative disease or neurodegenerative dementia comprising determining the level of one or more isoforms of at least one protein biomarker by any of the embodiments described above before treatment and at least one time during or following treatment and wherein successful treatment is demonstrated by the level of the said isoform(s) remaining stable or reverting to more normal levels. This is particularly beneficial in the assessment of experimental treatments for neurodegenerative dementia such as in human clinical trials. In an alternative embodiment of this aspect of the invention the monitoring of said isoform(s) may be used to guide selection of the optimal treatment for an individual patient wherein continued evolution of a disease biomarker profile indicates failure of current treatment and the need to provide an alternative treatment.

In a further aspect the present invention provides a neurodegenerative dementia determining system comprising a neurodegenerative dementia scoring apparatus, including a control component and a memory component, and an information communication terminal apparatus, said apparatuses being communicatively connected to each other via a network;

wherein the information communication terminal apparatus comprises:
1a) a clusterin glycoform profile data sending unit that transmits measured glycoform profile data of a subject to the neurodegenerative dementia scoring apparatus; and
1b) an evaluation result-receiving unit that receives the evaluation result of the neurodegenerative dementia score of the subject transmitted from the neurodegenerative dementia scoring apparatus;
   and wherein the control component comprises:
2a) a clusterin glycoform profile data-receiving unit that receives clusterin glycoform profile data of the subject from the information communication terminal apparatus;
2b) a clusterin glycoform profile matching unit that determines the closeness of fit of the clusterin glycoform profile data of the subject with a reference clusterin glycoform profile data record stored in the memory unit;
2c) a neurodegenerative dementia score-determining unit that determines the neurodegenerative dementia score of the subject based on the closeness of fit calculated by the clusterin glycoform profile matching unit; and
2d) a determination result-sending unit that transmits the neurodegenerative dementia score of the subject obtained by the neurodegenerative dementia score-determining unit to the information communication terminal apparatus.

In some cases, said clusterin glycoform profile comprises the relative proportions in a sample, e.g. a plasma sample, of the subject of at least 5, 6, 7, 8, 9, or at least 10 glycopeptides as set forth in Table 1A or 1B.

In a further aspect the present invention provides a method for identifying agents to be evaluated for therapeutic efficacy against a neurodegenerative disease or dementia, comprising: contacting a β-N-acetyl-glucosaminidase with a suitable substrate in the presence of a test agent and in the absence of the test agent and comparing the rate or extent of β-N-acetyl-glucosaminidase activity in the presence and in the absence of the test agent, wherein a test agent that inhibits β-N-acetyl-glucosaminidase activity is identified as an agent to be evaluated for therapeutic efficacy against a neurodegenerative dementia. In particular, the method may further comprise evaluating the test agent for the ability to reduce or block dementia-driven glycan pruning of tetra-antennary glycoforms of human clusterin protein or a glycosylated fragment thereof.

In a further aspect of the invention there is provided a method of identifying protein modifying enzymes such as glycotransferases and glycosidases that are active in disease. Such enzymes may serve as novel therapeutic targets and may provide alternative means for diagnosis and prognostic monitoring of disease.

Thus, a method of diagnosis of the presence or stage of dementia is provided comprising the measurement of the activity of glycosidases or glycotransferases present in a sample of tissue or bodily fluid taken from a subject suspected of having dementia on an artificial glycopeptide or glycotransferase substrate wherein the truncation or complete removal of antennary glycan structures on the glycopeptide's or glycotransferase's substrate are detected.

Several circulating glycoproteins are known to be associated with dementia (Nuutinen, Suuronen et al. 2009; Sato, Endo 2010; Butterfield, Owen et al. 2011). Clusterin in CSF for example has been linked to the mechanism of beta amyloid protein clearance whilst cellular clusterin is believed to mediate cellular signaling in response to toxic beta amyloid in neurons (Killick, Ribe et al. 2012). Alterations in the type and extent of N-linked glycosylation is known to affect protein function and stability and alterations in the distribution of circulating clusterin glycoforms may significantly affect its function in clearing aggregated proteins such a beta amyloid in Alzheimer's disease.

Thus in a further aspect of the invention methods of treating neurodegenerative disease or dementia by the administration of inhibitors of β-N-acetyl-glucosaminidase are provided. Such inhibitors prevent the “accelerated aging” of functional glycoproteins through loss of glycan antennae, enabling such glycoproteins to retain their normal function. Accordingly, the present invention also provides a method of treating neurodegenerative dementia by the administration to a subject diagnosed with dementia of a therapeutic amount of an inhibitor of β-N-acetyl-glucosaminidase. In a related aspect, the present invention provides an inhibitor of β-N-acetyl-glucosaminidase for use in a method of treatment of neurodegenerative disease or dementia in a mammalian subject.

The invention will now be described in more detail, by way of example and not limitation, by reference to the accompanying drawings. Many equivalent modifications and variations will be apparent to those skilled in the art when given this disclosure. Accordingly, the exemplary embodiments of the invention set forth are considered to be illustrative and not limiting. Various changes to the described embodiments may be made without departing from the scope of the invention. All documents cited herein are expressly incorporated by reference.

### BRIEF DESCRIPTION OF THE FIGURES

FIG. 1 Theoretical and actual clusterin glycoform distributions in 2DE. Panel A—Mathematical modeling of Clusterin alpha and beta chain Additional series for Tetra-antennary structures can be modeled in a similar manner (not shown). Panel B—2DE spots of immune-precipitated protein. 16 distinct spots were analysed and fully sialylated N-glycans were the most abundant structure at each glycosylation site in all 16 spots. The shift in pI observed for different gel spots is most likely driven by glycosylation via alterations in number of antennae and sialic acids.

FIG. 2. Tabular representation of individual glycoforms of four clusterin peptides detected in 16 spots on 2DE gels (Peptide A: SEQ ID NO: 2; Peptide B: SEQ ID NO: 11; Peptide C: SEQ ID NO: 7; Peptide D: SEQ ID NO: 10).

FIG. 3. Vector diagram illustrating the change in 2DE coordinates associated with the removal of specific glycan units from N linked carbohydrates

FIG. 4. Structure of the NA3 substrate (Dextra-UK Ltd, Catalogue No: C1124) Molecular Weight=2006.82 Da Chemical Formula: C₇₆H₁₂₇N₅O₅₆

FIG. 5. ESI-TOF spectrum of intact NA3 substrate showing presence of doubly charged molecular ion at m/z 1003.87 and related sodium and potassium cations.

FIG. 6. MS/MS spectrum of m/z 1050.74 the [M+3H]³⁺ molecular ion for clusterin glycopeptide of molecular weight 3149.22 Da. The fragment ions enable the structure of the glycan to be deduced with the fragment ion at m/z 574.47 representing the [Peptide+HexNac]+ moiety. Hence the sequence of the “naked” peptide is HN*STGCLR (SEQ ID NO: 2) and a fully sialylated bi-antennary glycan structure, SA₂-(HexNAc-Hex)₂, is attached to the asparagine residue (N*)

FIG. 7. Mass spectrum showing molecular ions for two sialylated forms of the tetra-antennary glycopeptides HN*STGCLR (SEQ ID NO: 2) observed in instances of low atrophy and not observed in high atrophy.

FIG. 8. Relative percentage of tetra-antennary glycoforms within eight individuals with low and high levels of hippocampal atrophy.

FIG. 9. Shows box plots of significantly regulated clusterin β64N glycopeptides from Discovery Cohort (Orbitrap Fusion) A) β64N_SA1-(HexNAc-Hex)2-core; B) β64N_SA2-(HexNAc-Hex)2-core; C) β64N_SA1-(HexNAc-Hex)3-core; D) β64N_SA2-(HexNAc-Hex)3-core; E) β64N_SA3-(HexNAc-Hex)3-core; and F) βB4N_SA3-(HexNAc-Hex)4-core.

FIG. 10. Shows box plots of significantly regulated clusterin β64N glycopeptides from Replication Cohort (Orbitrap Fusion) A) β64N_SA1-(HexNAc-Hex)2-core; B) β64N_SA1-(HexNAc-Hex)3-core; and C) β64N_SA2-(HexNAc-Hex)3-core.

FIG. 11. Shows box plots of significantly regulated clusterin β64N glycopeptides from combined Discovery and Replication Cohorts (Orbitrap Fusion) A) β64N_SA1-(HexNAc-Hex)2-core; B) β64N_SA2-(HexNAc-Hex)2-core; C) β64N_SA1-(HexNAc-Hex)3-core; and D) β64N_SA2-(HexNAc-Hex)3-core.

FIG. 12. Shows box plots of significantly regulated clusterin β64N glycopeptides from Discovery Cohort by SRM analysis (TSQ Vantage) A) β64N_SA1-(HexNAc-Hex)2-core; B) β64N_SA2-(HexNAc-Hex)2-core; C) β64N_SA1-(HexNAc-Hex)3-core; D) β64N_SA2-(HexNAc-Hex)3-core; and E) β64N_SA1-(HexNAc-Hex)4-core.

FIG. 13. Shows an SDS-PAGE image of albumin/IgG-depleted normal human plasma. Red bars represent cut points and numbers represent the band number used for Orbitrap analysis to identify clusterin glycopeptides.

FIG. 14. Shows total ion chromatogram (TIC) of band #4-#9 from depleted plasma using glyco-SRM method. Eight clusterin β64N glycopeptides were served as precursors (m/z 953.71, 1050.74, 1075.42, 1172.45, 1197.13, 1269.49, 1294.17, 1391.53), and fragment ions at m/z 366.14, 574.56, and 657.24 were set as transition ions for each precursor.

FIG. 15. Shows XIC of band #7 presenting various retention time and peak area of eight glycoforms at site β64N.

FIG. 16. Shows box plots of significantly regulated clusterin β64N glycopeptides from combined Discovery & Validation Cohorts by SRM analysis (TSQ Vantage) A) β64N_SA1-(HexNAc-Hex)2-core; B) β64N_SA2-(HexNAc-Hex)2-core; C) β64N_SA1-(HexNAc-Hex)3-core; D) β64N_SA2-(HexNAc-Hex)3-core; E) β64N_SA1-(HexNAc-Hex)4-core; F) β64N_SA3-(HexNAc-Hex)3-core; G) β64N_SA2-(HexNAc-Hex)4-core; and H) β64N_SA3-(HexNAc-Hex)4-core.

FIG. 17. Shows Table 1A—The Clusterin GlycoMod database v1.0 (control plasma) (SEQ ID NOs: 2-7 & 10, respectively).

FIG. 18. Shows Table 1B—The Clusterin GlycoMod database v1.1 (MCI/AD plasma) (SEQ ID NOs: 2-7, 9, 10, & 8 respectively).

### DETAILED DESCRIPTION

In describing the present invention, the following terms will be employed, and are intended to be defined as indicated below.

The term “subject” includes a human or an animal. In accordance with certain embodiments of the present invention, the subject may have been previously diagnosed with AD and/or previously diagnosed with mild cognitive impairment (MCI). The subject is preferably a human. The subject may be a human of at least 60 years of age, optionally at least 70 or at least 80 years of age.

The term “diagnosis”, as used herein, includes the provision of any information concerning the existence, non-existence or probability of the disorder in a patient. It further includes the provision of information concerning the type or classification of the disorder or of symptoms which are or may be experienced in connection with it. This may include, for example, diagnosis of the severity of the disorder. It encompasses prognosis of the medical course of the disorder, for example its duration, severity and the course of progression from MCI to Alzheimer's disease.

Currently disease status is assessed by duration of disease from inception to present (longer duration equals more severe disease) and clinical assessment measures. These assessment measures include clinical tests for memory and other cognitions, clinical tests for function (abilities of daily living) and clinical assessments of global severity. Trials of potential therapies in AD are currently evaluated against these measures. The FDA and other medicines approval bodies require as part of these assessments measures of both cognition and global function. The Global Dementia Scale is one such measure of global function. It is assessed by later assessment of severity including cognition and function against a standardised set of severity criteria.

The term “alleviate”, as used herein, in relation to Alzheimer's disease means any form of reducing one or more undesired symptoms or effects thereof. Any amelioration of Alzheimer's disease of the patient falls within the term “alleviation”. Amelioration may also include slowing down the progression of the disease.

As used herein “assessing” AD includes the provision of information concerning the type or classification of the disease or of symptoms which are or may be experienced in connection with it. This specifically includes prognosis of the medical course of the disease, for example its duration, severity and the course and rate of progression from e.g. MCI or pre-symptomatic AD to clinical AD. This also includes prognosis of AD-associated brain pathology such as fibrillar amyloid burden, cortical and hippocampal atrophy and accumulation of neurofibrillary tangles. The assessment may be of an aggressive form of AD and/or a poor prognosis.

As used herein “biological sample” refers to any biological liquid, cellular or tissue sample isolated or obtained from the subject. In accordance with the present invention the “protein-containing sample” may be any biological sample as defined herein. The biological sample may, in certain cases, comprise blood plasma, blood cells, serum, saliva, urine, cerebro-spinal fluid (CSF) or a tissue biopsy. The biological sample may have been stored (e.g. frozen) and/or processed (e.g. to remove cellular debris or contaminants) prior to determining the amount (e.g. concentration) of the at least one protein isoform and/or glycoform in question that is found in the sample.

### Exemplary Glycoform Analysis—Clusterin

Clusterin (Apolipoprotein J; SP-40,40; TRPM-2; SGP-2; pADHC-9; CLJ; T64; GP III; XIP8) is a highly conserved disulfide-linked secreted heterodimeric glycoprotein of 75-80 kDa but truncated forms targeted to nucleus have also been identified. The protein is constitutively secreted by a number of cell types including epithelial and neuronal cells and is a major protein in physiological fluids including plasma, milk, urine, cerebrospinal fluid and semen.

Preferably, clusterin comprises or consists of an amino acid sequence having at least 70%, 80%, 90%, 95%, 99% or 100% identity to the human clusterin sequence disclosed in UniProt Accession No. P10909, sequence version 1 and GI No. 116533 (SEQ ID NO: 1) (incorporated herein by reference in its entirety), calculated over the full length of said human clusterin sequence; or a fragment thereof comprising at least 5, 10, 15, 20, 30, 50, 100, 150, 200, 250, 300, 350, 400, 425 or 449 contiguous amino acids.

Expression of the clusterin gene is significantly elevated in Alzheimer's disease (AD) brain (May et al., 1990) and levels of plasma clusterin have also been shown to correlate with AD progression (Thambisetty et al., 2010). The inventors have previously identified several plasma clusterin isoforms as candidate biomarkers for AD using 2-dimensional gel electrophoresis (2DE).

However, the use of immunoassays and unmodified peptides in selected reaction monitoring (SRM) experiments did not fully replicate the regulation seen in 2DE. The inventors hypothesised that this disconnect is perhaps due to alterations in specific post-translational events that were not being replicated in the validation studies. Clusterin is a highly-glycosylated secreted protein and because glycosylation plays an important role in physiological functions of clusterin (Stuart et al., 2007) the inventors proposed that the detailed profiling of plasma clusterin and comparison of glycosylation profiles observed in distinct clinically classified subjects, for example patients with low or high atrophy of the hippocampus, may reveal more potent biomarker isoforms.

Guided by the observations relating to the clusterin glycoforms, which demonstrate a β-N-acetyl-glucosaminidase activity in plasma, the inventors also devised a novel assay using a defined substrate to measure this specific activity.

### Example 1

### Gel Electrophoresis Analysis of Plasma Clusterin Isoforms

Methods

Human clusterin, was enriched by immunoprecipitation (IP) from albumin/IgG-depleted plasma, using a monoclonal anti-clusterin antibody (Millipore). Immunoprecipitated proteins were first analysed by Western blotting as a quality control, then separated by either two-dimensional electrophoresis (2DE) or SDS-PAGE. The spots and single band (#3) of interest were excised, reduced, alkylated and digested in-gel with trypsin prior to analysis by mass spectrometry (MS). Samples were analysed via LC-MS/MS using nanoflow reverse phase chromatography (EASY-nLC II, ThermoFisher Scientific) and a Top20 collision induced dissociation (CID) method (Orbitrap Velos, ThermoFisher Scientific). Glycopeptides were manually identified by the presence of glycan-specific oxonium ion fragments, m/z 204.08 for N-acetylhexosamine, [HexNAc]⁺, m/z 366.14 for hexose-N-acetylhexosamine, [Hex-HexNAc]⁺, and m/z 657.24 for N-acetylneuraminic acid-hexose-N-acetylhexosamine [NeuAc-Hex-HexNAc]⁺ in the MS/MS spectra.

Results

2DE Spots

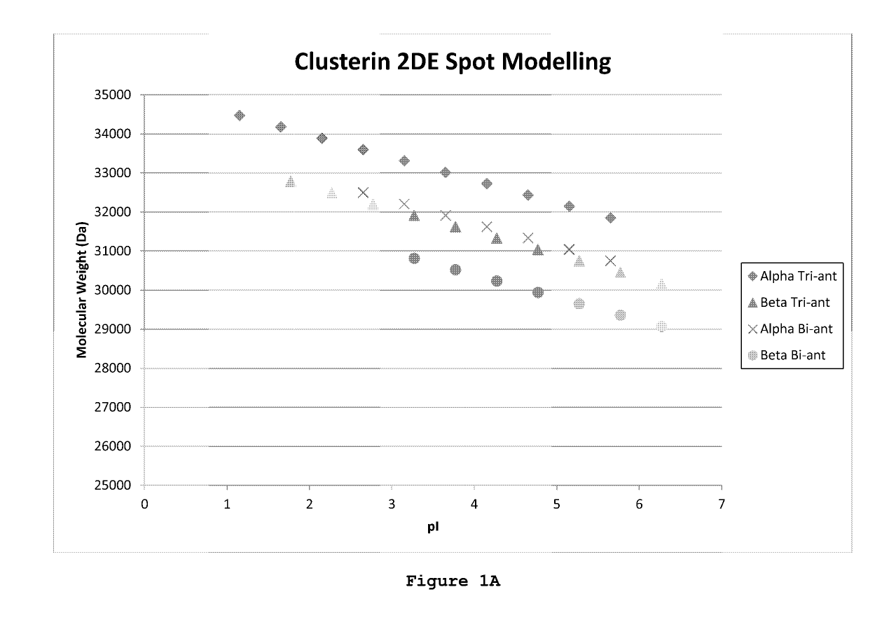

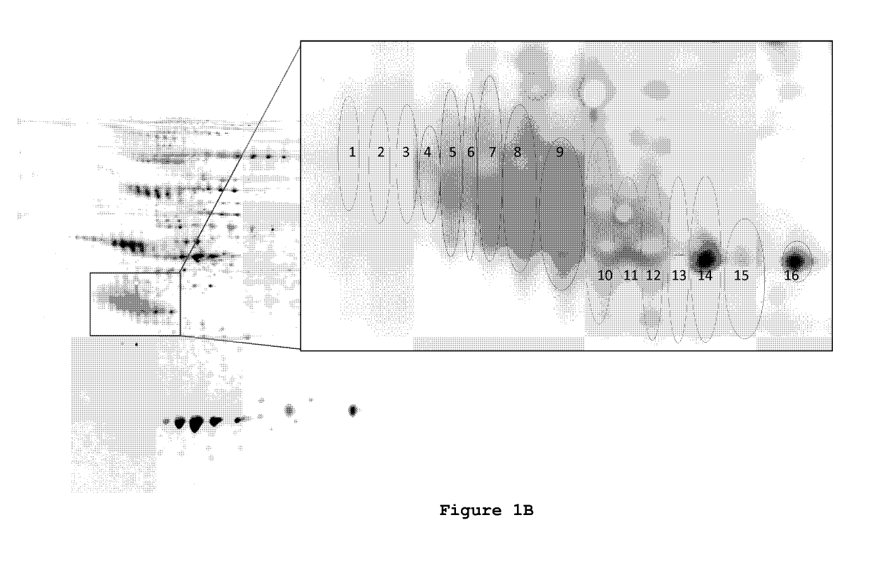

Initially, mathematical modelling was used to create an artificial map of the various clusterin glycoforms for the separate alpha and beta chains (FIG. 1A). Further refinement of this approach was used to classify individual clusterin related glycoforms using simple (x, y) coordinates. In this way, the inventors were able to predict the content of the individual 2DE spots, and demonstrate that discrete coordinates are shared by multiple glycoforms. Hence, 2DE spots are likely to be composite mixtures containing several glycoforms. This information was then used to aid the interpretation of complex LC/MS/MS data, which subsequently provided some rather useful insights.

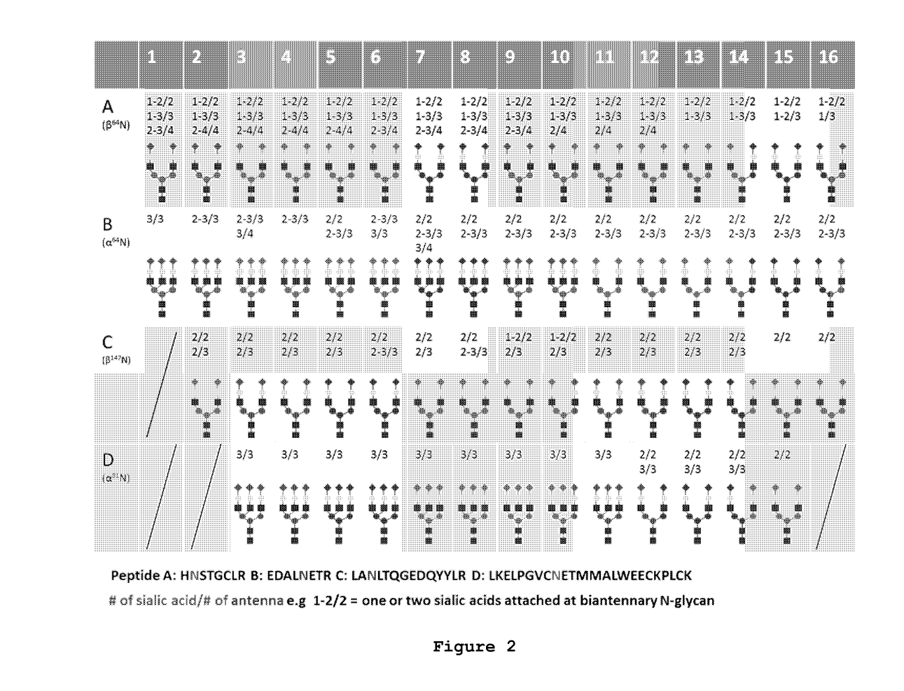

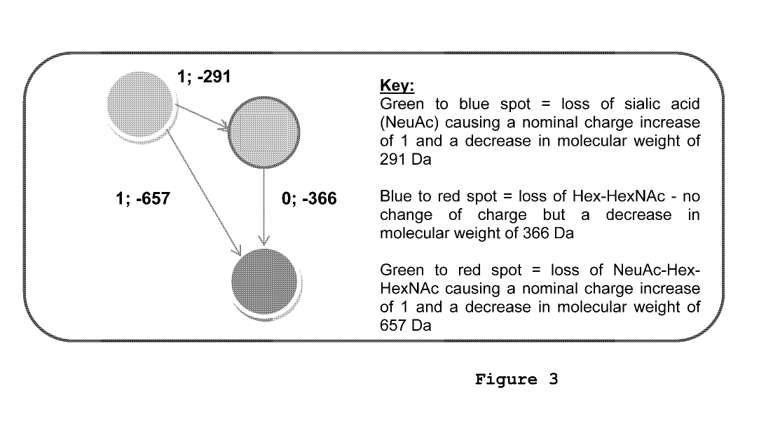

Firstly, it became apparent that the major components within each of the 2DE spots were, without exception, always fully sialylated forms, being either tetra, tri or biantennary structures (FIG. 1B). This was surprising because it had originally been predicted that differences in sialic acid were the primary cause of the separation of distinct forms during electrophoresis, with loss of 291 Da concurrent with a decreasing charge thus resulting in a shift towards a more basic iso-electric point. However, the LC/MS/MS results (FIG. 2) indicated a trend towards lower number of antennae and this suggested the successive removal of whole antennae as a more pronounced effect on the location of the 2DE spots. A basic vector was devised to illustrate this phenomena (FIG. 3) and it transpires that the detected clusterin glycoforms actually now indicate evidence to support both the removal of sialic acids alone as well as removal of full antennae suggesting distinct neurominidase and β-N-acetyl-glucosaminidase activity respectively.

### Example 2

### Design of a Substrate Assay to Measure Specific n-Acetyl-Glucosaminidase Activity in Plasma

The results of glycan analysis of 16 different clusterin isoforms visible on 2-dimensional gel electrophoresis showed a sequential removal of sialic acids and entire antennae. Several of the truncated glycoforms appeared to correlate with clusterin protein spots previously identified as candidate biomarkers of AD and MCI. Until now no detailed analysis of glycosylation of these clusterin isoforms has been performed and it was surprising to discover that the majority of the disease associated modification in plasma clusterin could be accounted for by the activity of a single glycosidase, namely β-N-acetyl-glucosaminidase.

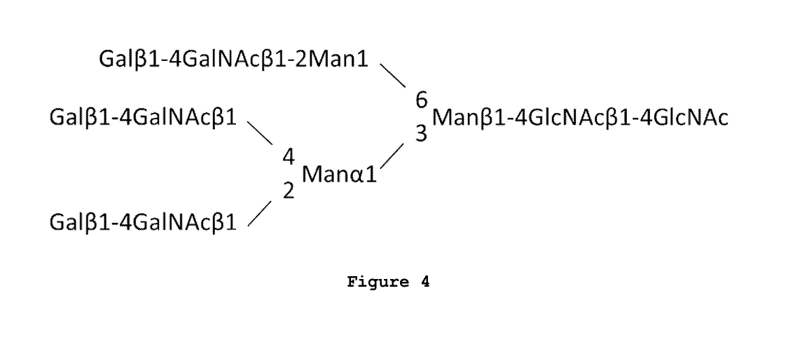

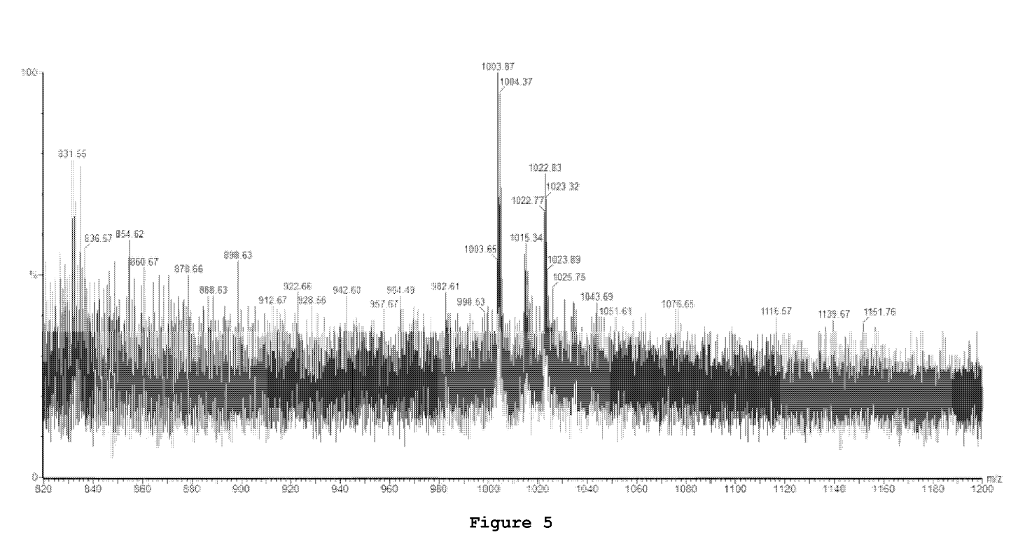

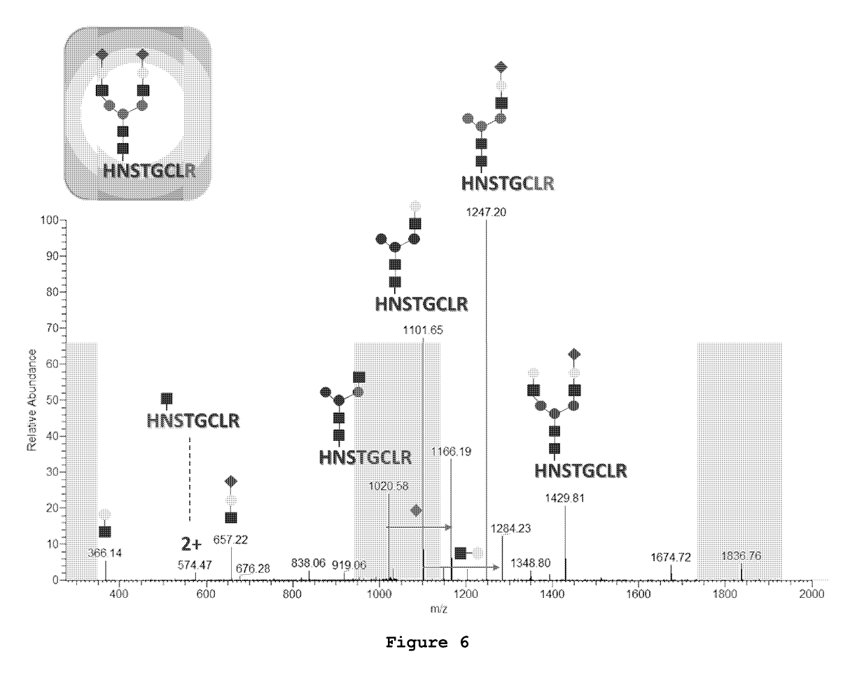

The inventors thus set up a specific assay method to determine the activity of β-N-acetyl-glucosaminidase in tissue or bodily fluid samples taken from subjects suspected of having, or previously diagnosed with MCI, AD or other dementia. The artificial glycan NA3 substrate (FIG. 4) contains both β1,2 and β1,4 linkages between adjoining β-N-acetyl-glucosamine and mannose subunits and is a preferred substrate to differentiate β1,2 and β1,4 N-acetyl-glucosaminidase activity in plasma. The molecular weight of the substrate is 2006.82 Da and an [M+2H]²⁺ ion is detected at m/z 1003.8 using an ESI-TOF mass spectrometer (FIG. 6).

NA3 substrate is added to an appropriate sample of tissue or body fluid from a subject suspected of having, or previously diagnosed with dementia to achieve a final concentration of 300-1,000 pg/μl and incubated at 37° C. for 4-24 hours. The test sample is then centrifuged to remove debris and an aliquot submitted to LC-MS/MS analysis. The measurement of molecular ions corresponding to loss of either two or one antennae indicate β1,2 and β1,4 N-acetyl-glucosaminidase activity respectively.

### Example 3

### Analysis of Immuno-Precipitated Clusterin to Create a Unique Glycopeptide Reference Resource for Clusterin:_the Clusterin GlycoMod Database v1.0

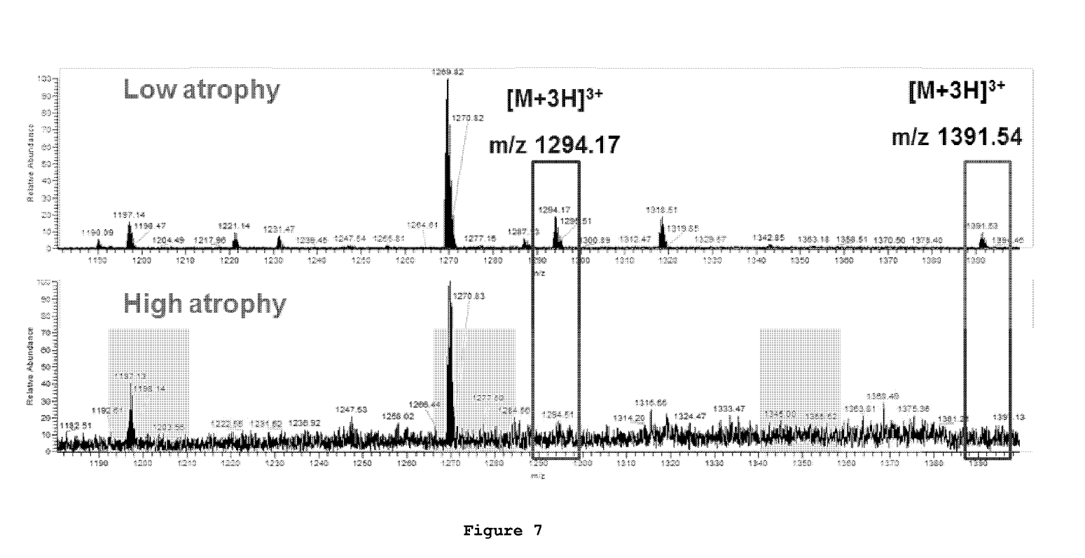

A representative pooled clinical plasma sample was used to develop methodology and to assemble an “observation-based” database containing 41 distinct glycoforms associated with anticipated glycosylation consensus sites within the amino acid sequence. For each glycopeptide the m/z charge state and retention time (RT) of the analyte was tabulated (see Table 1A). Unambiguous annotation of the glycopeptide required the detection of the [Peptide+HexNAc]⁺ fragment ion in the corresponding MS/MS spectra and interpretation of additional fragment ions relating to the sequential dissociation of the individual glycan subunits. An example MS/MS spectrum is shown (FIG. 7) and the current iteration of the Clusterin GlycoMod database is provided in Table 1A. An updated iteration of the Clusterin GlycoMod database is provided in Table 1B.

Using immuno-precipitation and LC/MS/MS we have characterised 41 glycopeptides encompassing 5 of 6 anticipated N-linked glycosylation consensus sites in plasma clusterin. In total 41 different N-linked glycopeptides have been characterised and are listed herein. The glycan distribution at these 5 sites was consistent with a CV of <15% (n=3 from two plasma samples) indicating the technical and biological reproducibility of the method.

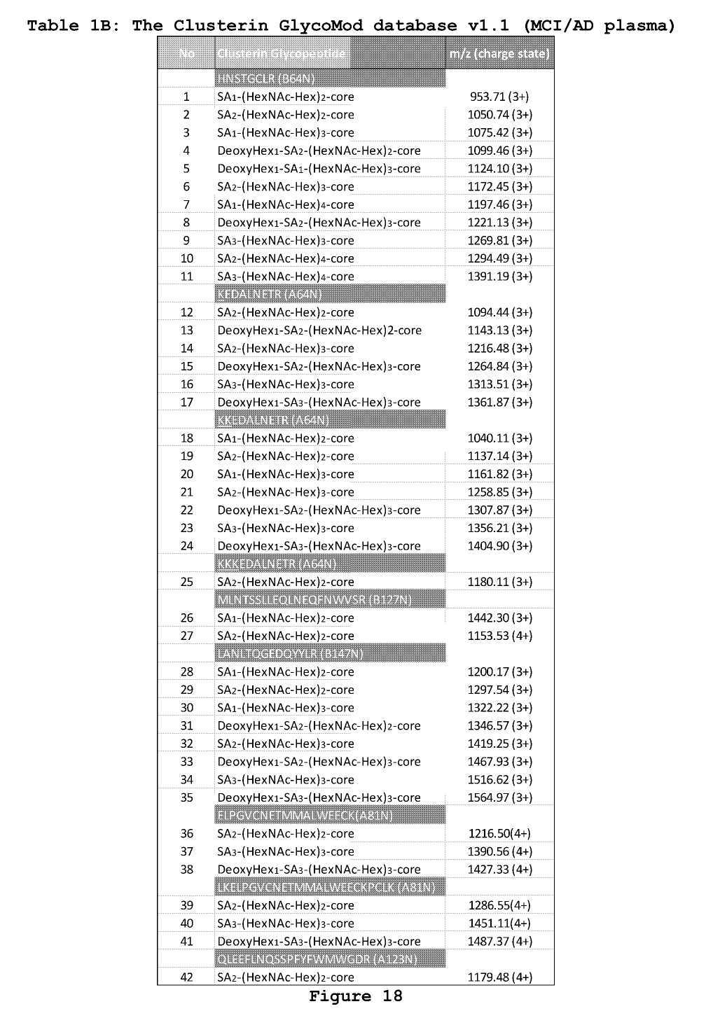

The inventors have previously demonstrated 5 of 6 predicted N-linked glycosylation sites within human plasma clusterin (GlycoMod database v1.0). It would be understood by the skilled practitioner that expansion of the GlycoMod database to cover all N-linked and O-linked sites of all the protein biomarkers is within the scope of the present invention. Indeed, the inventors have subsequently completed mapping of the sixth N-linked site in human clusterin as set out in Table 1B (FIG. 18), showing clusterin glycopeptides of version 1.1 of the clusterin GlycoMod database (MCI/AD plasma).

### Example 4

### Analysis of Immuno-Precipitated Clusterin to Compare Individuals with Low and High Atrophy

The inventors have identified certain isoforms of clusterin as differentially regulated in the plasma of patients with AD relative to non-demented controls. Furthermore, it has also been shown that certain spots comprising clusterin on 2DE gels correlate with the level of hippocampal atrophy, whilst yet other isoforms correlated with the subsequent rate of disease progression in AD.

The inventors obtained plasma samples from four subjects previously diagnosed with AD who had a low level of hippocampal atrophy and from four subjects previously diagnosed with AD with high hippocampal atrophy. Clusterin was enriched using immunoprecipitation, and subjected to the LC-MS/MS method described above. Surprisingly, they identified that the extent of glycan pruning correlated with hippocampal atrophy. In patients with low levels of hippocampal atrophy there was little evidence of pruning of plasma clusterin. Conversely, plasma clusterin from subjects with high levels of hippocampal atrophy was typically pruned to remove one or more complete antennae within the N-linked glycans.

As an example, two sialylated forms of the tetra-antennary glycopeptide HN*STGCLR (SEQ ID NO: 2) are observed as triply charged molecular ions at m/z 1391 and m/z 1294.17 but only in individuals with low atrophy (FIG. 7). These moieties are therefore potential alternative biomarkers concordant with the extent of hippocampal atrophy.

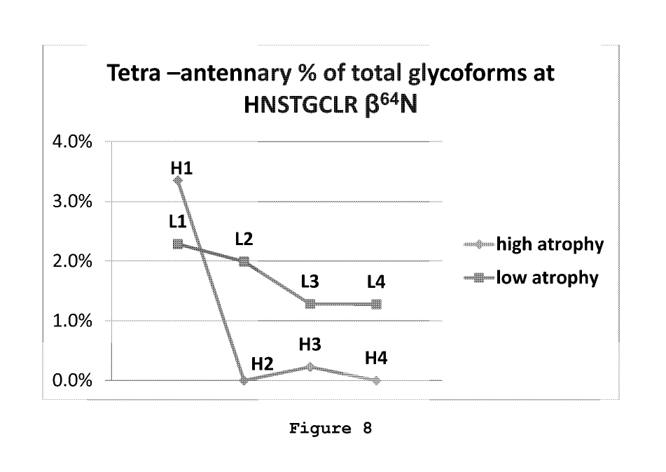

Using data from all N-linked glycans monitored by the LC-MS/MS method the inventors saw a consistent reduction in the level of tetra-antennary glycans in subjects with high levels of hippocampal atrophy compared to those with low levels. Based on the total glycoform signal for the N-linked glycosylation site on the tryptic peptide HN*STGCLR (SEQ ID NO: 2) of the clusterin beta chain (FIG. 8).

### Example 5

### Validation Analysis of Immuno-Precipitated Clusterin to Compare Individuals with Low and High Atrophy

Having identified that changes in Clusterin glycosylation patterns correlate to the extent of atrophy within a small cohort of clinical samples we performed a further validation study on an additional cohort of Alzheimer's disease patients with known levels of hippocampal atrophy. Additional bioinformatics approaches were also assessed for their impact on class segregation based on glycoform profiles and a new, higher sensitivity mass spectrometer was employed in the expectation of identifying additional diagnostic glycoforms of clusterin. To ensure correlation with earlier data, samples from the original 4×4 cohort (Discovery Cohort) used in Example 4 were re-analysed using the new methods alongside a separate cohort of 20 new samples from AD (n=10) and matched controls (n=10)(Replication Cohort). All sample details are provided in Table 2.

Sample Cohort Details

TABLE 2
Sample details associated with 4 vs 4 and 10 vs 10 cohorts

| | | | | | Mean | |
|---|---|---|---|---|---|---|
| Study | Disease | | | Mean | hippocampus | |
| ID | group | Gender | Age | Clusterin | (×10e−6) | Atrophy |
| 4.1 | AD | Female | 82 | 87 | 135 | High |
| 4.2 | MCI | Male | 79 | 93 | 264 | Low |
| 4.3 | MCI | Male | 72 | 90 | 274 | Low |
| 4.4 | MCI | Male | 75 | 90 | 264 | Low |
| 4.5 | AD | ND | ND | 153 | 161 | High |
| 4.6 | AD | Female | 78 | 84 | 164.5 | High |
| 4.7 | AD | Male | 69 | 90 | 106.5 | High |
| 4.8 | MCI | Female | 71 | 114 | 307.5 | Low |
| 10.1 | AD | Male | 79 | 154.32 | 111.5 | High |
| 10.2 | AD | Female | 76 | 337.7 | 124.0 | High |
| 10.3 | AD | Male | 77 | 422.11 | 125.5 | High |
| 10.4 | AD | Male | 69 | 252.19 | 233.0 | Low |
| 10.5 | AD | Female | 87 | 322.05 | 238.0 | Low |
| 10.6 | AD | Male | 71 | 289.22 | 228.0 | Low |
| 10.7 | AD | Female | 70 | 253.82 | 234.2 | Low |
| 10.8 | AD | Male | 70 | 303.13 | 0.0 | High |
| 10.9 | AD | Female | 83 | 530.31 | 136.5 | High |
| 10.10 | AD | Female | 65 | 497.37 | 227.1 | Low |
| 10.11 | AD | Female | 77 | 404.6 | 235.5 | Low |
| 10.12 | AD | Female | 76 | 241.56 | 99.6 | High |
| 10.13 | AD | Male | 76 | 300.21 | 109.5 | High |
| 10.14 | AD | Female | 67 | 323.54 | 135.0 | High |
| 10.15 | AD | Female | 72 | 280.5 | 228.0 | Low |
| 10.16 | AD | Female | 63 | 309.15 | 244.7 | Low |
| 10.17 | AD | Male | 83 | 423.18 | 127.0 | High |
| 10.18 | AD | Female | 71 | 7147.63 | 237.4 | Low |
| 10.19 | AD | Female | 68 | 307.14 | 140.0 | High |
| 10.20 | AD | Male | 79 | 351.02 | 237.5 | Low |

Methods

Clusterin was enriched from each sample, as described above. The relevant protein band was excised, reduced, alkylated, and digested with Trypsin. After clean up, the clusterin digests were split into two aliquots and each tested by nanoflow high performance liquid chromatography and Orbitrap Velos Pro or ultra-high performance liquid chromatography and Orbitrap Fusion Tribrid LC-MS/MS systems (all equipment from Thermo Scientific, Hemel Hempstead, UK). Data were ostensibly similar but, as expected, more glycosylated clusterin peptides were identified on the Fusion and so all subsequent analysis was performed on the Fusion dataset.

Bioinformatics

Mass spectrometer raw data were processed using Proteome Discoverer software (Thermo Scientific). Ion intensities for the glycosylated clusterin peptides and their fragments described in Tables 1A and 1B were exported into an Excel (Microsoft Corp) spreadsheet. We employed a sum scaling technique to normalise the data and calculated significance values (p) for each glycopeptide by comparing the median values between the low and high atrophy groups in the Discovery Cohort, Replication Cohort and a combined analysis of both Cohorts as a single group. Student's T test was used to identify peptide-associated glycoforms that change significantly between high and low atrophy, resulting in one-tailed p-values for each glycopeptide (see Tables 3A, 3B and 3C).

Results

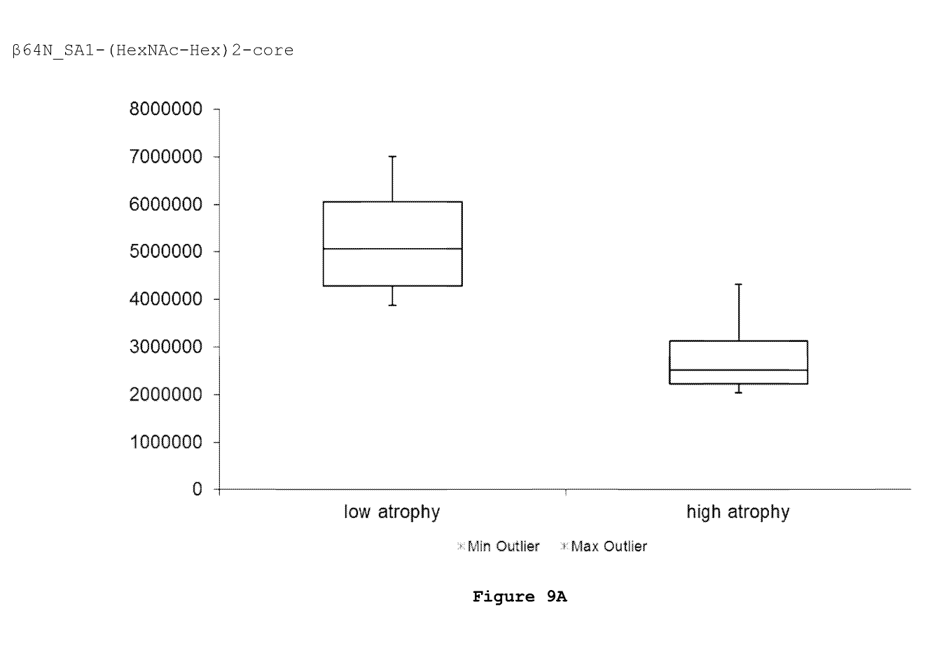

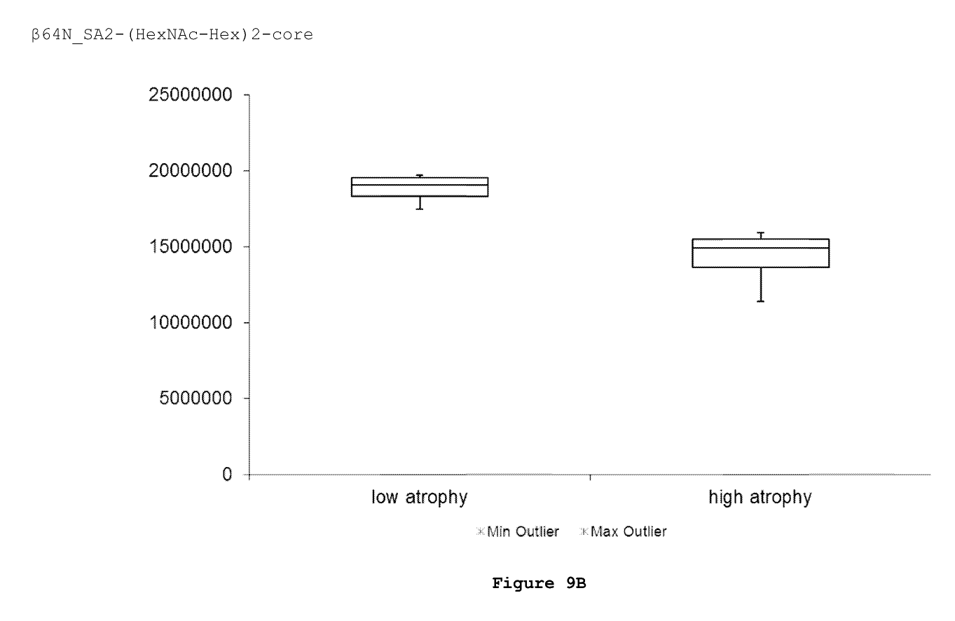

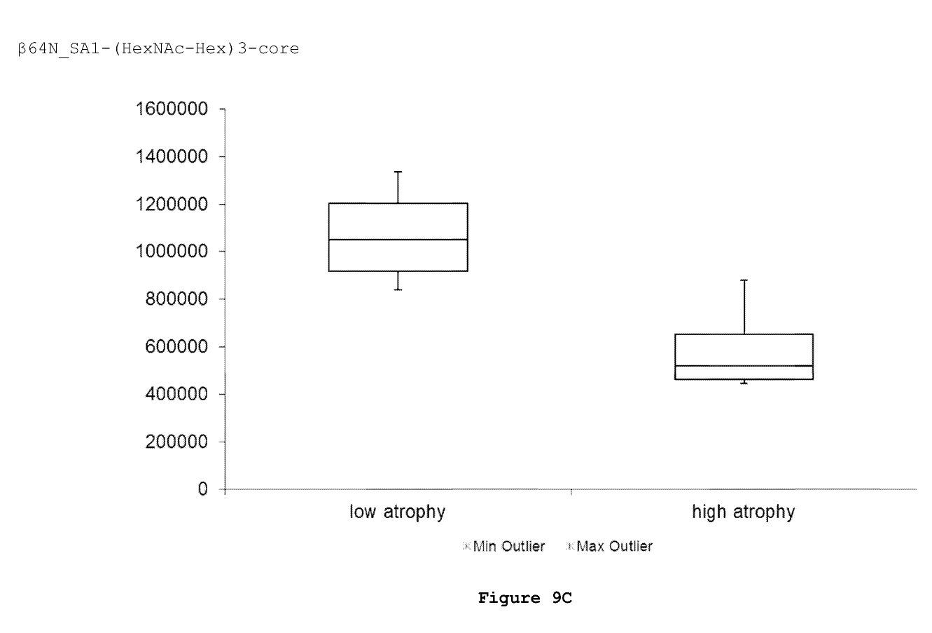

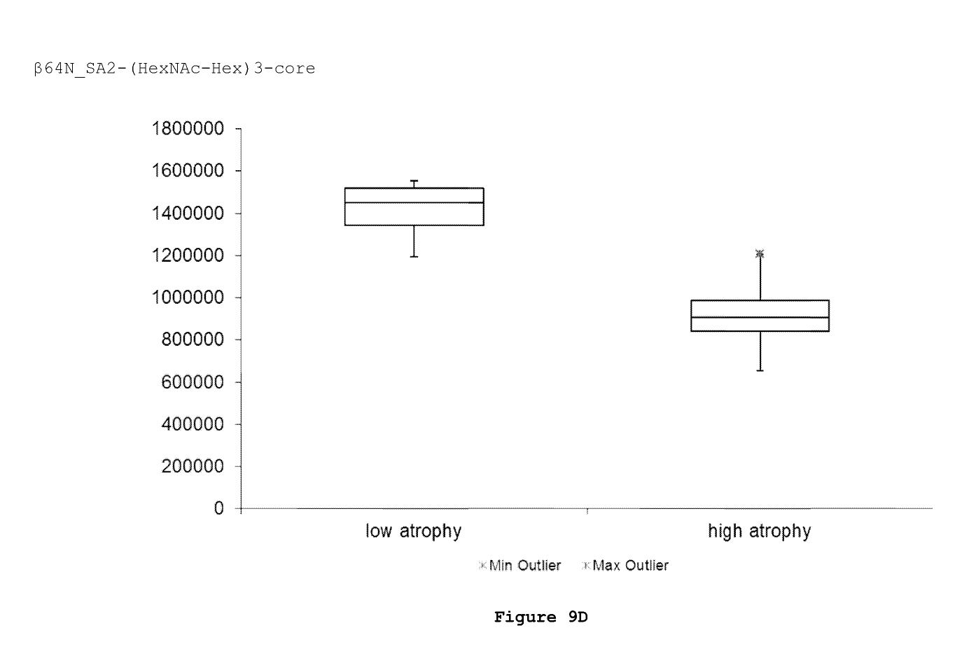

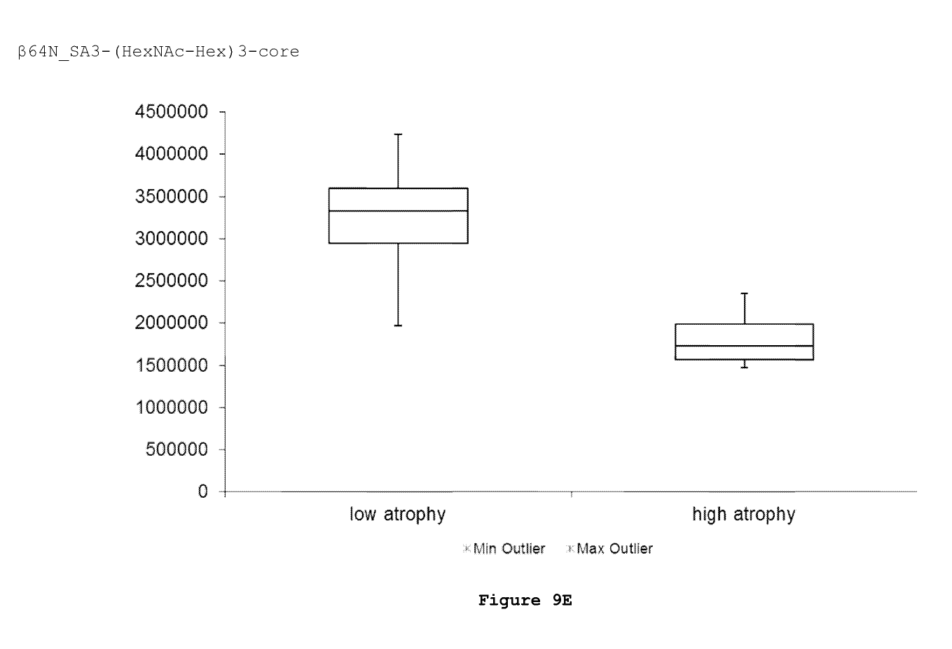

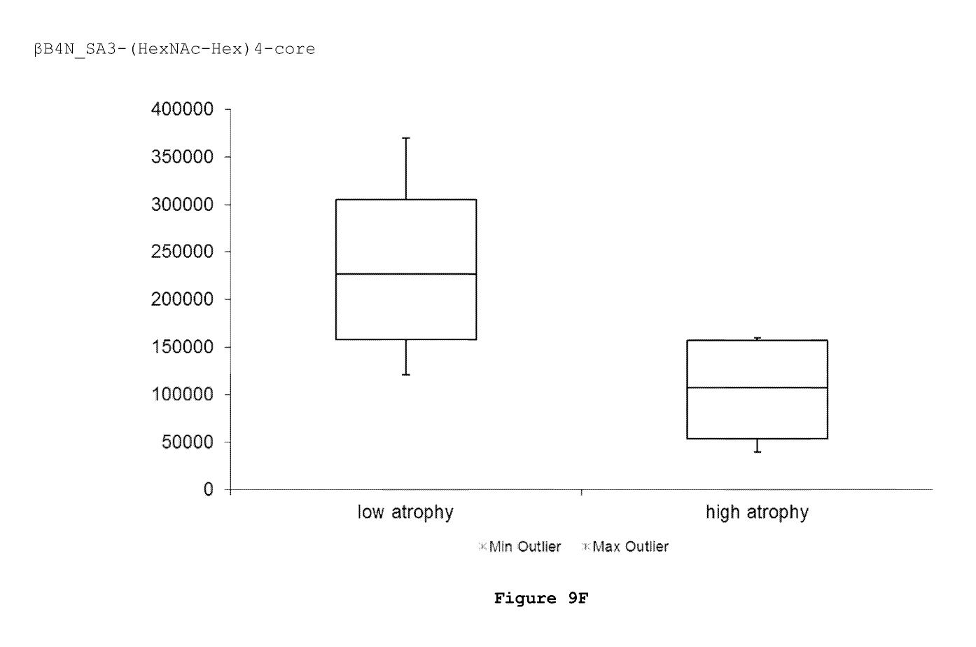

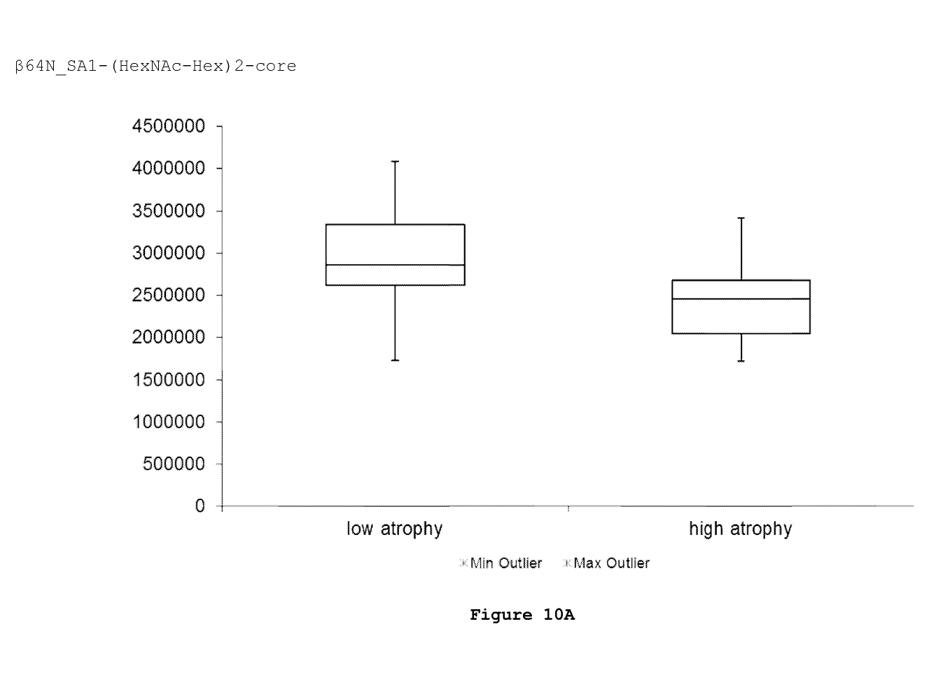

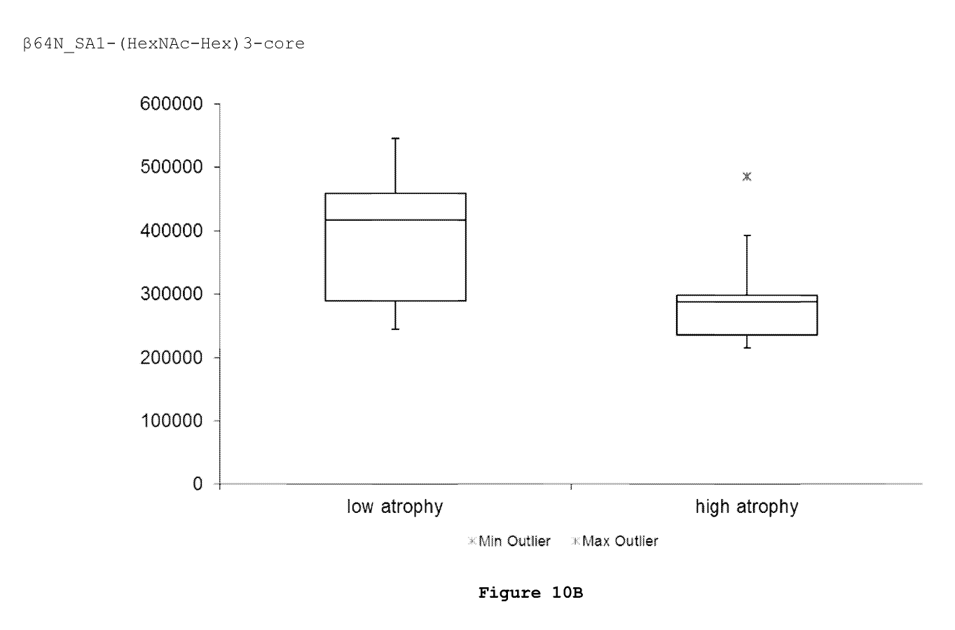

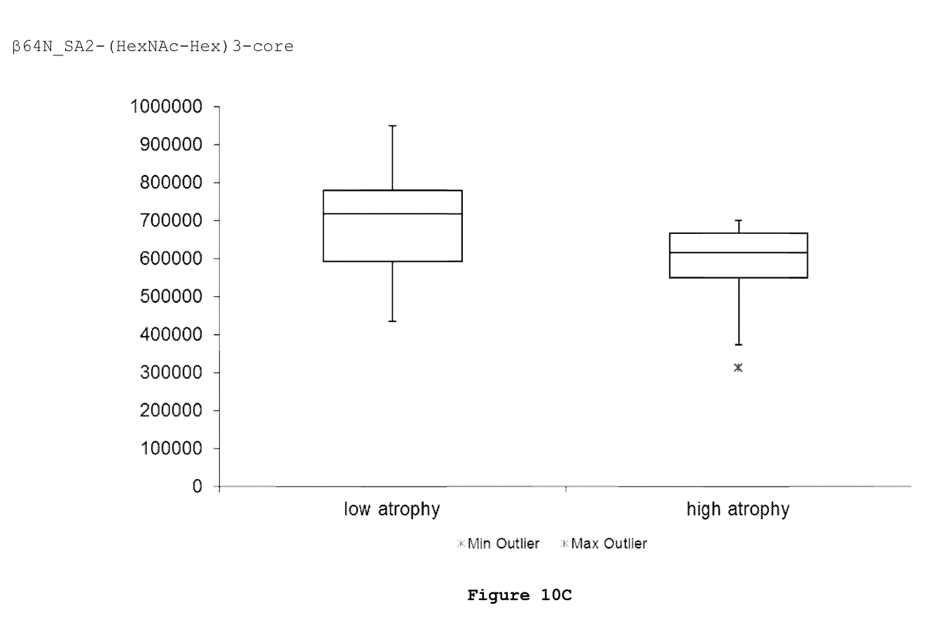

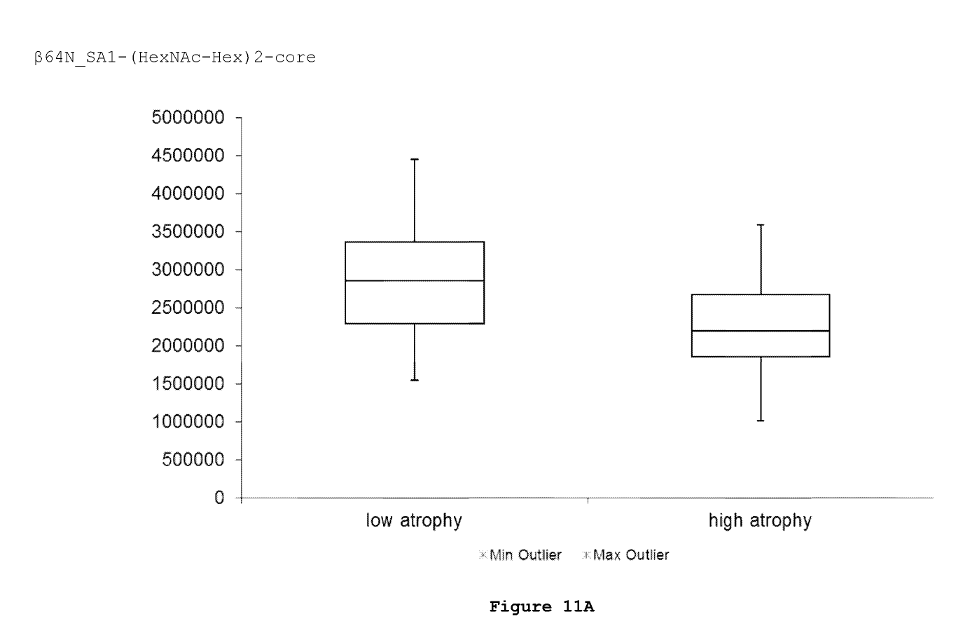

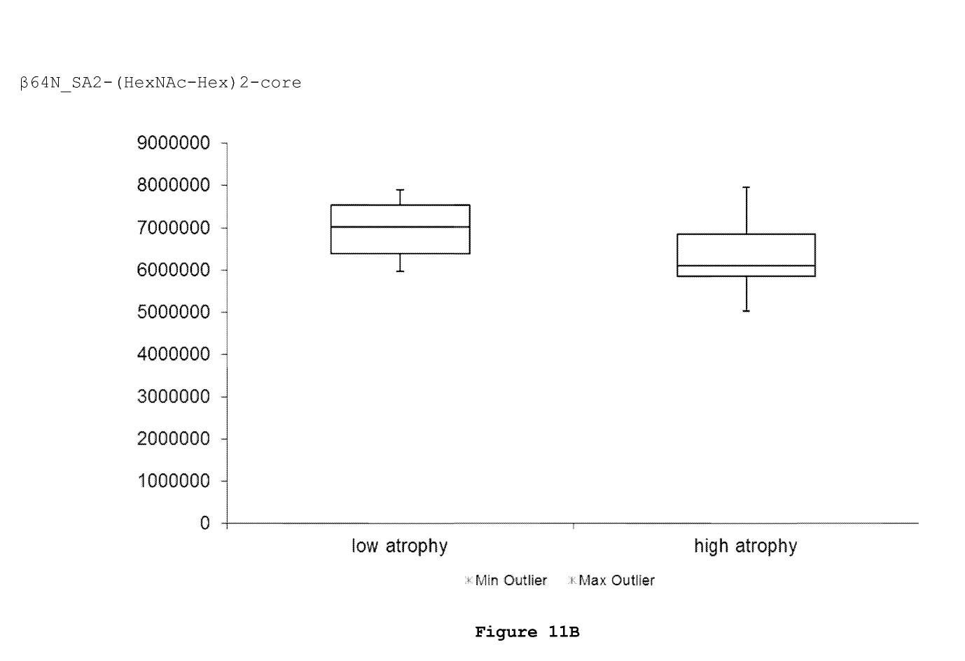

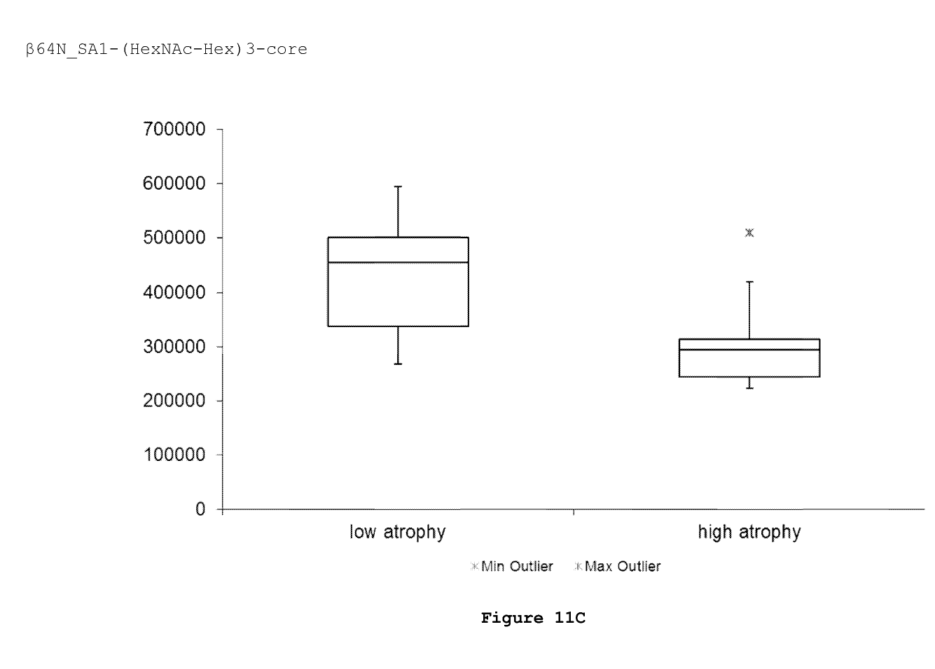

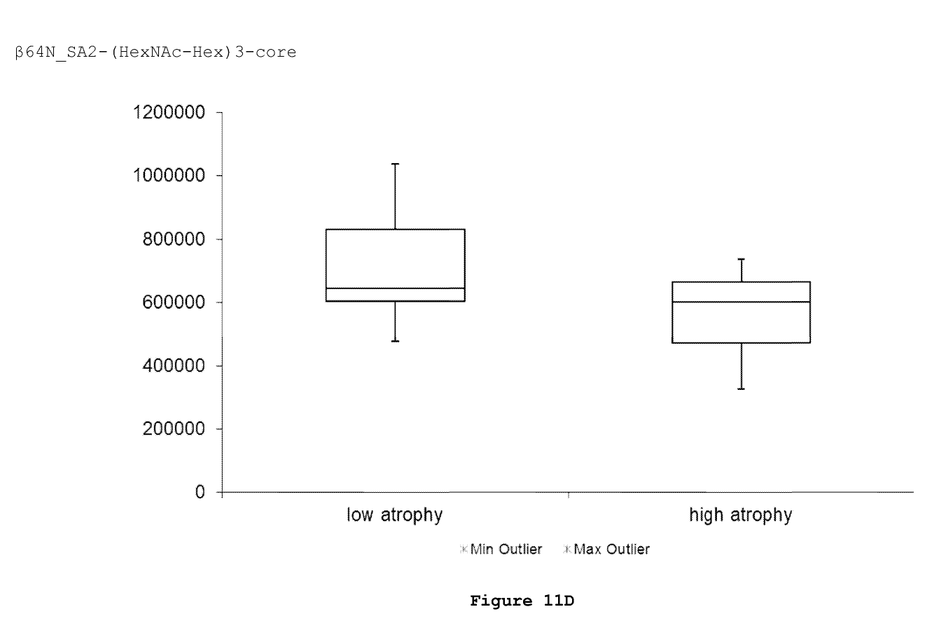

Using our IP-LC/MS/MS workflow on the Orbitrap Fusion Tribrid we were able to extend coverage to all six known N-glycosylation sites of clusterin: α64N, α81N, α123N, β64N, β127N, and β147N. By monitoring the glycan specific fragments we were also able to assign various antennary structures at all six sites and to perform relative quantification based on total ion counts. In total 42 different glycan structures were detected. Whilst most glycosylation sites showed no regulation in glycan structures between high and low levels of hippocampal atrophy, two sites—β64N and β147N—showed significant regulations between the clinical groups. The specific glycan structures showing significant (p≦0.05) changes between the clinical groups in the Discovery, Replication and combined Cohort analyses are indicated in Table 3A, 3B, and 3C respectively. Box plots for each glycopeptide were created to illustrate the separation achieved between the two groups (FIGS. 9-11).

Interestingly, six glycoforms at β64N glycosylation site HN*STGCLR (SEQ ID NO: 2) were found significantly decreased in the 4 high atrophy samples (Alzheimer's) compared to the 4 low atrophy samples (mild cognitive impairment) of the Discovery Cohort when measured on the Orbitrap Fusion. This included the sialylated forms of the tetra-antennary glycopeptide observed as triply charged molecular ions at m/z 1391.54 which was consistent with the previous Velos data analysis, confirming the robustness of this glycoform as a diagnostic marker to differentiate mild cognitive impairment from Alzheimer's disease when measured on a different LC-MS/MS platform.

In the larger replication cohort, three glycoforms of β64N glycopeptides were significantly reduced in high atrophy samples. These include the SA1-(HexNAc-Hex)2, SA1-(HexNAc-Hex)3 and SA2-(HexNAc-Hex)3 glycoforms seen at m/z 953.71, 1075.42, 1172.45 in the spectra. As all of these glycoforms were also seen reduced in high atrophy patients in the Discovery Cohort this further supports their utility as prognostic biomarkers in patients with confirmed Alzheimer's disease.

When the results of the two cohorts were combined we again, saw that changes in glycoforms found at site β64N correlated with atrophy, with four glycoforms significantly reduced over high atrophy, e.g. SA1-(HexNAc-Hex)2, SA2-(HexNAc-Hex)2, SA1-(HexNAc-Hex)3, and SA2-(HexNAc-Hex)3 at m/z 953.71, 1050.74, 1075.42, and 1172.45 respectively.

TABLE 3A
Significant changes in Clusterin glycopeptides (4 vs 4)

| M/Z (3+) | COMPOSITION | P-VALUE | SITE |
|---|---|---|---|
| 953.71 | SA1-(HexNAc-Hex)2-core | 0.016 | β64N |
| 1050.74 | SA2-(HexNAc-Hex)2-core | 0.003 | β64N |
| 1075.42 | SA1-(HexNAc-Hex)3-core | 0.009 | β64N |
| 1172.45 | SA2-(HexNAc-Hex)3-core | 0.006 | β64N |
| 1269.49 | SA3-(HexNAc-Hex)3-core | 0.017 | β64N |
| 1391.53 | SA3-(HexNAc-Hex)4-core | 0.043 | β64N |
| 1297.54 | SA2-(HexNAc-Hex)2-core | 0.044 | β147N |
| 1356.21 | SA3-(HexNAc-Hex)3-core | 0.044 | α64N |

TABLE 3B
Significant changes in Clusterin glycopeptides (9 vs 10)

| M/Z (3+) | COMPOSITION | P-VALUE | SITE |
|---|---|---|---|
| 953.71 | SA1-(HexNAc-Hex)2-core | 0.035 | β64N |
| 1075.42 | SA1-(HexNAc-Hex)3-core | 0.019 | β64N |
| 1172.45 | SA2-(HexNAc-Hex)3-core | 0.043 | β64N |
| 1297.54 | SA2-(HexNAc-Hex)2-core | 0.044 | β147N |
| 1137.14 | SA2-(HexNAc-Hex)2-core | 0.016 | α64N |
| 1356.21 | SA3-(HexNAc-Hex)3-core | 0.007 | α64N |

TABLE 3C
Significant changes in Clusterin glycopeptides (combined 13 vs 14)

| M/Z (3+) | COMPOSITION | P-VALUE | SITE |
|---|---|---|---|
| 953.71 | SA1-(HexNAc-Hex)2-core | 0.022 | β64N |
| 1050.74 | SA2-(HexNAc-Hex)2-core | 0.022 | β64N |
| 1075.42 | SA1-(HexNAc-Hex)3-core | 0.001 | β64N |
| 1172.45 | SA2-(HexNAc-Hex)3-core | 0.019 | β64N |

Conclusion

Use of the Orbitrap Fusion increased total glycoform coverage from 4 to 6 N-linked sites. Several β64N site glycoforms are significantly reduced in plasma of patients with Alzheimer's disease compared to individuals with mild cognitive impairment. Four of these glycoforms are also reduced in Alzheimer's patients with high levels of hippocampal atrophy. In combination this confirms the utility of clusterin isoforms as diagnostic and prognostic markers for Alzheimer's disease.

### Example 6

### Development and Preliminary Testing of a Selective Reaction Monitoring (SRM) Method for 8 Glycoforms of Clusterin in Human Plasma

In readiness for higher throughput measurements within much larger numbers of clinical samples, we have also developed a targeted Selective Reaction Monitoring (SRM) method to measure specific glycopeptides of Clusterin. This newly established TSQ-SRM workflow used eight glycoforms of Clusterin β64N glycopeptides as precursors, and two glycan-specific oxonium ion fragments at m/z 366.14 and m/z 657.24 as transitions (see Table 4). Additionally, the peptide ion at m/z 574.56 representing [HN*STGCLR]²⁺ (SEQ ID NO: 2) where N*=Asparagine residue+HexNac, was included to serve as the third transition ion providing confirmation of site-specific information. Details of each monitored transition is provided in Table 4.

TABLE 4
Glyco-SRM method of TSQ analysis.

| | | | SRM | | | | | |
|---|---|---|---|---|---|---|---|---|
| | | | colli- | | | Po- | | |
| | | Prod- | sion | Start | Stop | lar- | Trig- | Refer- |
| # | Parent | uct | energy | Time | Time | ity | ger | ence |
| 1 | 953.712 | 366.140 | 30 | 0 | 30 | + | 100 | No |
| 2 | 953.712 | 574.556 | 30 | 0 | 30 | + | 100 | No |
| 3 | 953.712 | 657.235 | 30 | 0 | 30 | + | 100 | No |
| 4 | 1050.744 | 366.140 | 33 | 0 | 30 | + | 100 | No |
| 5 | 1050.744 | 574.556 | 33 | 0 | 30 | + | 100 | No |
| 6 | 1050.744 | 657.235 | 33 | 0 | 30 | + | 100 | No |
| 7 | 1075.423 | 366.140 | 34 | 0 | 30 | + | 100 | No |
| 8 | 1075.423 | 574.556 | 34 | 0 | 30 | + | 100 | No |
| 9 | 1075.423 | 657.235 | 34 | 0 | 30 | + | 100 | No |
| 10 | 1172.454 | 366.140 | 37 | 0 | 30 | + | 100 | No |
| 11 | 1172.454 | 574.556 | 37 | 0 | 30 | + | 100 | No |
| 12 | 1172.454 | 657.235 | 37 | 0 | 30 | + | 100 | No |
| 13 | 1197.134 | 366.140 | 38 | 0 | 30 | + | 100 | No |
| 14 | 1197.134 | 574.556 | 38 | 0 | 30 | + | 100 | No |
| 15 | 1197.134 | 657.235 | 38 | 0 | 30 | + | 100 | No |
| 16 | 1269.487 | 366.140 | 41 | 0 | 30 | + | 100 | No |
| 17 | 1269.487 | 574.556 | 41 | 0 | 30 | + | 100 | No |
| 18 | 1269.487 | 657.235 | 41 | 0 | 30 | + | 100 | No |
| 19 | 1294.165 | 366.140 | 42 | 0 | 30 | + | 100 | No |
| 20 | 1294.165 | 574.556 | 42 | 0 | 30 | + | 100 | No |
| 21 | 1294.165 | 657.235 | 42 | 0 | 30 | + | 100 | No |
| 22 | 1391.532 | 366.140 | 45 | 0 | 30 | + | 100 | No |
| 23 | 1391.532 | 574.556 | 45 | 0 | 30 | + | 100 | No |
| 24 | 1391.532 | 657.235 | 45 | 0 | 30 | + | 100 | No |

In previous studies (data not shown), we were able to extract clusterin glycopeptides from human serum without prior immunoprecipitation. Given the potential sensitivity gains offered by SRM methods we followed a more straightforward geLC method for clusterin enrichment which would be more compatible with high throughput analysis such as would be required for a clinical diagnostic.

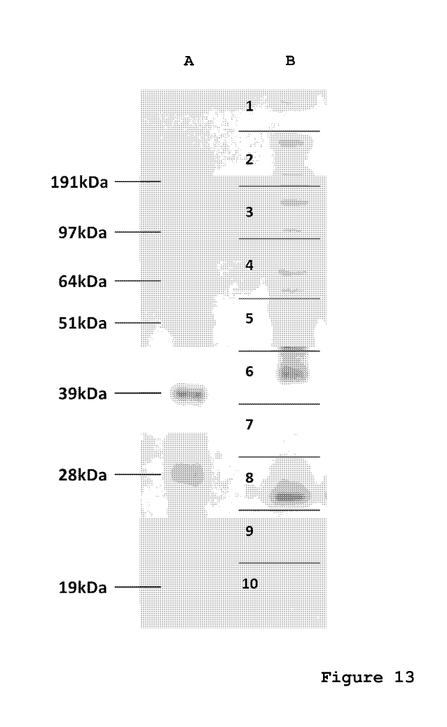

Initially, to identify the location of clusterin in a one dimensional SDS-Polyacrylamide gel electrophoresis (SDS-PAGE) experiment normal human plasma (Dade-Behring, Germany) was depleted of albumin and IgG and proteins extracted in Laemmli buffer and subjected to SDS-PAGE. FIG. 13 shows the Gel-10 analysis of albumin/IgG-depleted plasma. All ten bands were excised, reduced, alkylated, and trypsin-digested prior to MS analysis on an Orbitrap Velos Pro. Glycopeptides of clusterin were identified in the tryptic digests of bands #5, #6, and #7 (data not shown) with the majority seen in band #7 with 45% of peptide coverage.

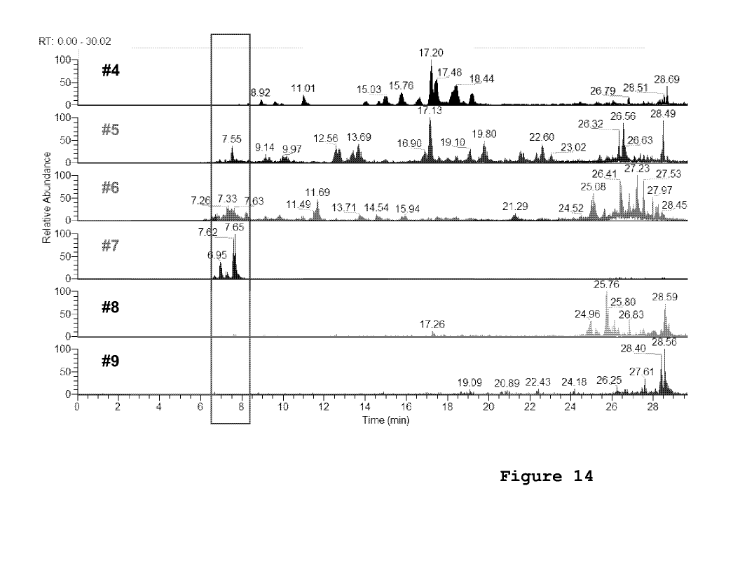

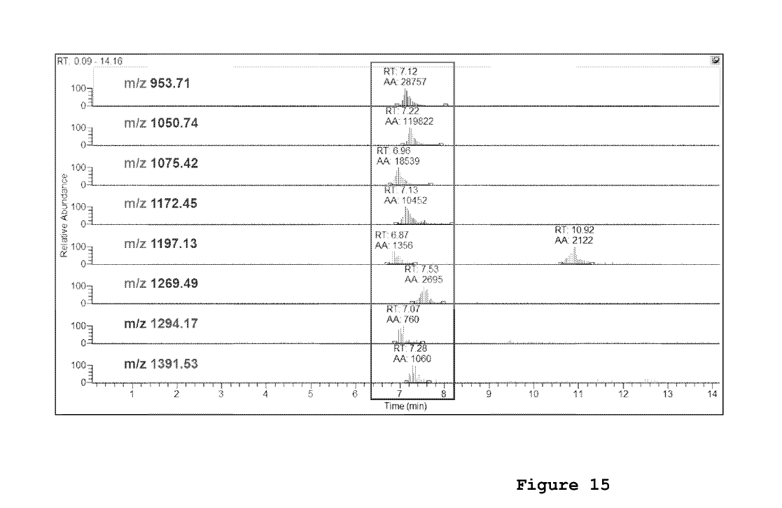

All ten tryptic-digested gel bands were also submitted for analysis using the newly-developed clusterin glycoform SRM method to confirm the suitability of the geLC method for sample preparation. FIG. 14 shows Clusterin glycopeptides were found in the band #5, #6 and #7, and majority of Clusterin was identified at band #7, suggesting these glyco-SRM results were consistent with the previous Orbitrap Velos Pro discovery data. When the SRM data files were examined for the extracted ion chromatograms (XIC) of eight β64N glycopeptide precursors (FIG. 15), we were able to determine that the majority of them eluted between 7-8 minutes. Precise identification of elution time allows subsequent scheduling of SRM or adjustment of elution buffers to improve separation of closely related species if more complex methods should be developed subsequently. It is a particular advantage of the present method that the integrated peak area for each monitored species can be used for label-free quantification using a sum-scaled approach. Furthermore, since this Gel10-glyco-SRM method does not require immunoprecipitation and Clusterin glycopeptides can be detected within 30 minutes by TSQ, instead of one hour by Orbitrap, this newly-established method provides a more efficient and faster way to verify the potential biomarker glycopeptide of Clusterin. The same method may also be used for clinical assessment of patient samples to aid the diagnosis of MCI and Alzheimer's disease as well as providing prognostic information on the rate of hippocampal atrophy and cognitive decline. It would also be understood that the same SRM method may be applied with little further optimisation to digested human plasma, serum, saliva, urine or cerebrospinal fluid without the need for prior SDS-PAGE separation.

It is also possible to employ the same SRM method for the analysis of clusterin enriched from human plasma by immunoprecitpitation. Thus, to further validate our targeted biomarker glycopeptides, the clusterin glycol-SRM method was applied to evaluate immunoprecipitated clusterin from the Discovery Cohort. As expected, the SRM method gave tighter quantitative results and this improved precision resulted in higher levels of significance for the reduction in specific glycoforms in Alzheimer's patients with higher levels of hippocampal atrophy (Table 5). In total, five of the eight monitored glycopeptides at β64N were significantly reduced in high atrophy cases.

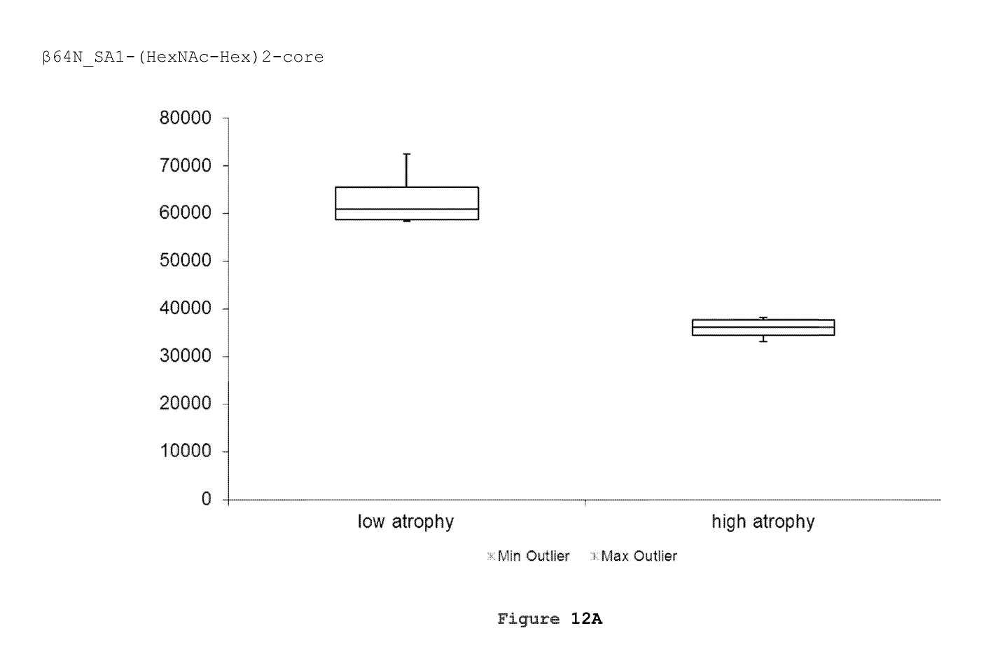

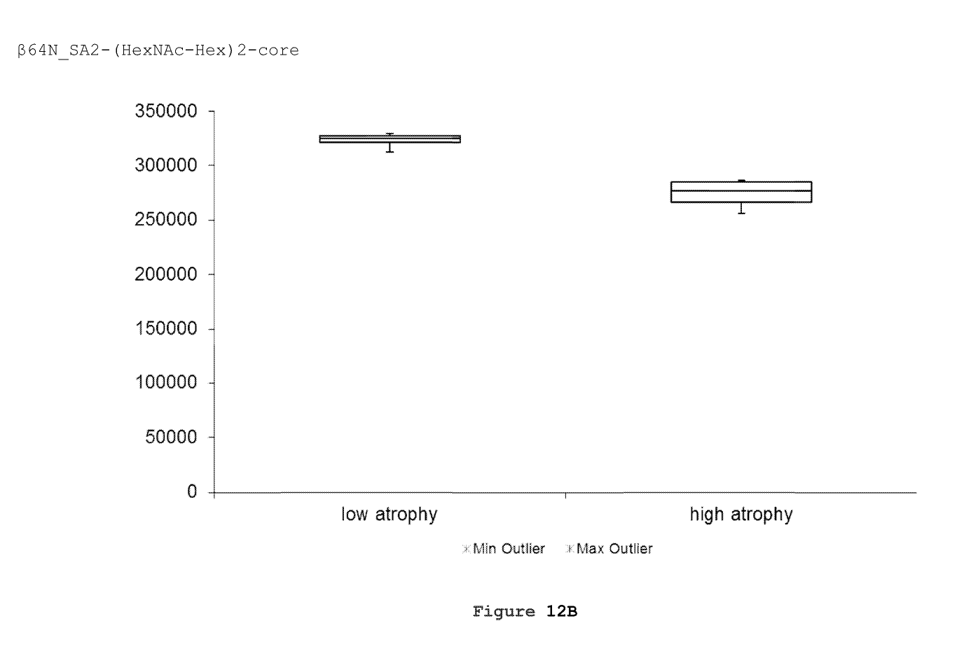

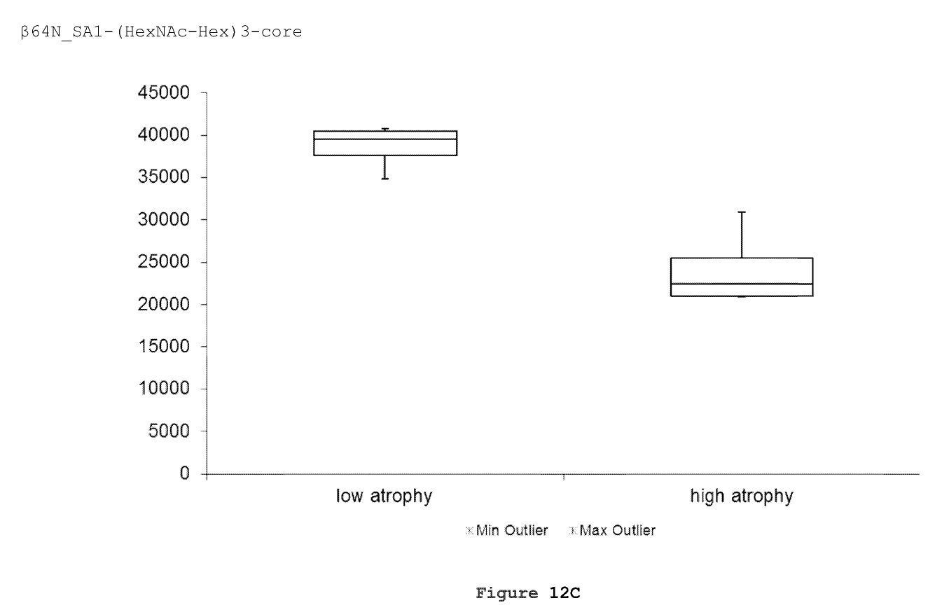

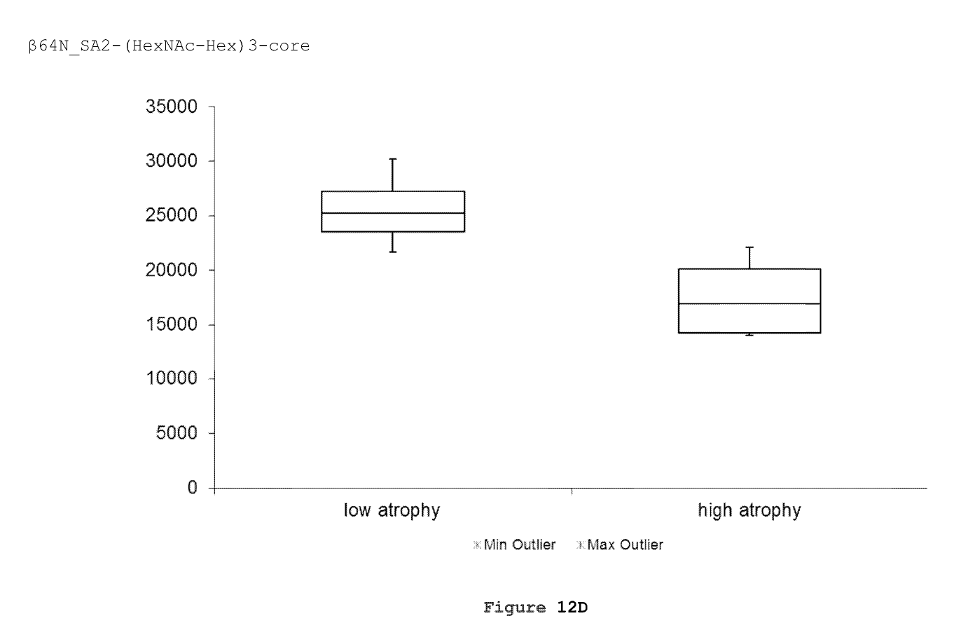

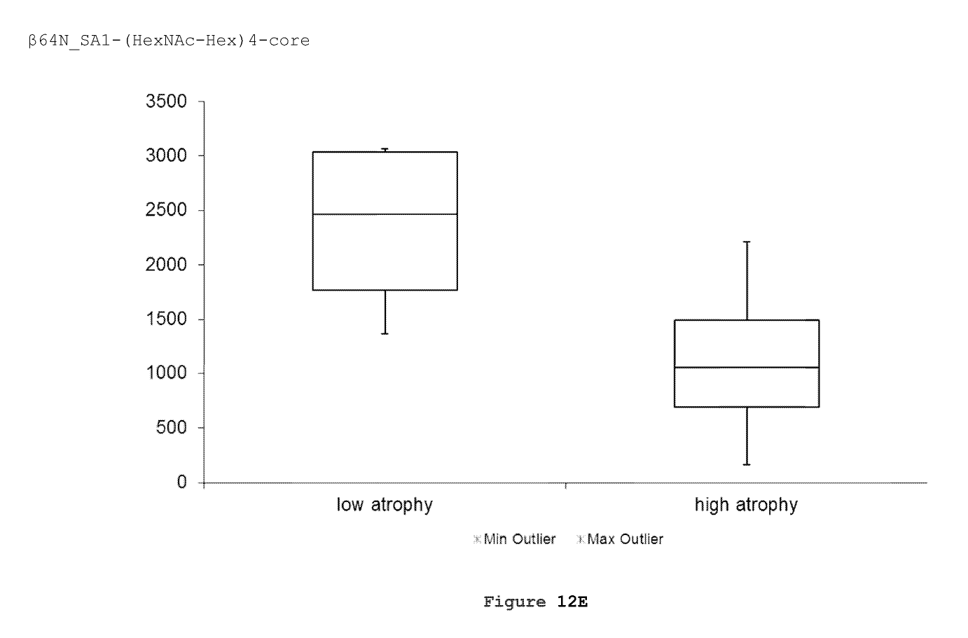

A selection of box plots for SRM quantification of individual clusterin glycoforms is provided in FIG. 12.

TABLE 5
Significant changes in Clusterin glycopeptides using
Gel10-glyco-SRM method (4 vs 4) Discovery cohort

| m/z (3+) | composition | p-value* | site |
|---|---|---|---|
| 953.71 | SA1-(HexNAc-Hex)2-core | 0.0001 | β64N |
| 1050.74 | SA2-(HexNAc-Hex)2-core | 0.0004 | β64N |
| 1075.42 | SA1-(HexNAc-Hex)3-core | 0.0009 | β64N |
| 1172.45 | SA2-(HexNAc-Hex)3-core | 0.012 | β64N |
| 1197.13 | SA1-(HexNAc-Hex)4-core | 0.044 | β64N |

*The p-value indicates significance of change between high and low atrophy groups.

### Example 7

### Validation Study of Gel10-Glyco-SRM Clusterin Glycoform Selected Reaction Monitoring Assay

The eight clusterin glycopeptide Gel10-glyco-SRM assay developed in Example 6 was applied to the analysis of the Validation Cohort of Alzheimer's disease patient plasma samples comprising 9 cases with [high] level of hippocampal atrophy and 10 cases with [low] level of hippocampal atrophy. Samples were as described in Table 2 and all sample preparation and analytical methods are as described in Example 6.

Across this cohort three specific β64N site-specific glycoforms showed a statistically significantly difference between patients with high levels of hippocampal atrophy and those with lower rates of hippocampal atrophy (Table 6).

TABLE 6
Performance of Gel10-glyco-SRM Clusterin
Glycoform Assay in the Validation Cohort

| m/z (3+) | Composition | p-value* | site |
|---|---|---|---|
| 953.71 | SA1-(HexNAc-Hex)2-core | 0.000964891 | β64N |
| 1050.74 | SA2-(HexNAc-Hex)2-core | 0.009457781 | β64N |
| 1172.45 | SA2-(HexNAc-Hex)3-core | 0.006985634 | β64N |

*The p-value indicates significance of change between high and low atrophy groups.

When the results for the Discovery and Validation Cohorts were combined, surprisingly all eight glycoforms attained statistical significance for reduced concentrations in the high atrophy group compared to those with low hippocampal atrophy (Table 7). The power of these eight clusterin glycopeptides to differentiate patients based on their hippocampal volume provides a minimally invasive means to diagnose and predict the progression of Alzheimer's disease and will be applicable to the analysis of other neurodegenerative diseases characterized by the aggregation of proteins leading to neuronal damage including Parkinson's Disease, Huntington's Disease, and Frontotemporal Dementia.

TABLE 7
Combined Performance of Gel10-glyco-SRM Clusterin Glycoform
SRM in combined Discovery and Validation Cohort

| m/z (3+) | composition | p-value* | site |
|---|---|---|---|
| 953.71 | SA1-(HexNAc-Hex)2-core | 2.81311E−05 | β64N |
| 1050.74 | SA2-(HexNAc-Hex)2-core | 5.67936E−10 | β64N |
| 1075.42 | SA1-(HexNAc-Hex)3-core | 0.000662932 | β64N |
| 1172.45 | SA2-(HexNAc-Hex)3-core | 8.03747E−08 | β64N |
| 1197.13 | SA1-(HexNAc-Hex)4-core | 0.002226471 | β64N |
| 1269.49 | SA3-(HexNAc-Hex)3-core | 0.001689634 | β64N |
| 1294.17 | SA2-(HexNAc-Hex)4-core | 0.001327899 | β64N |
| 1391.53 | SA3-(HexNAc-Hex)4-core | 0.009999 | β64N |

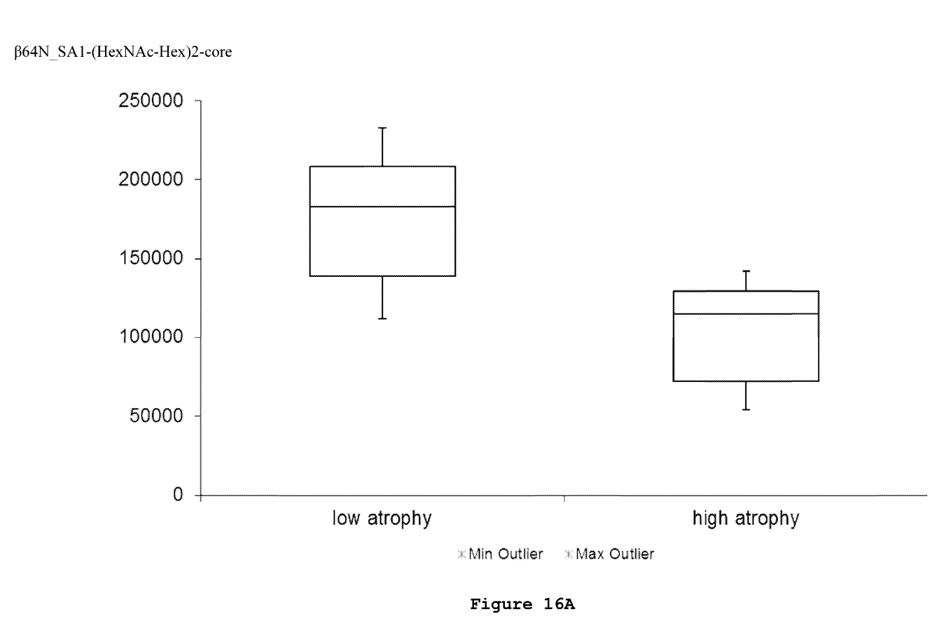

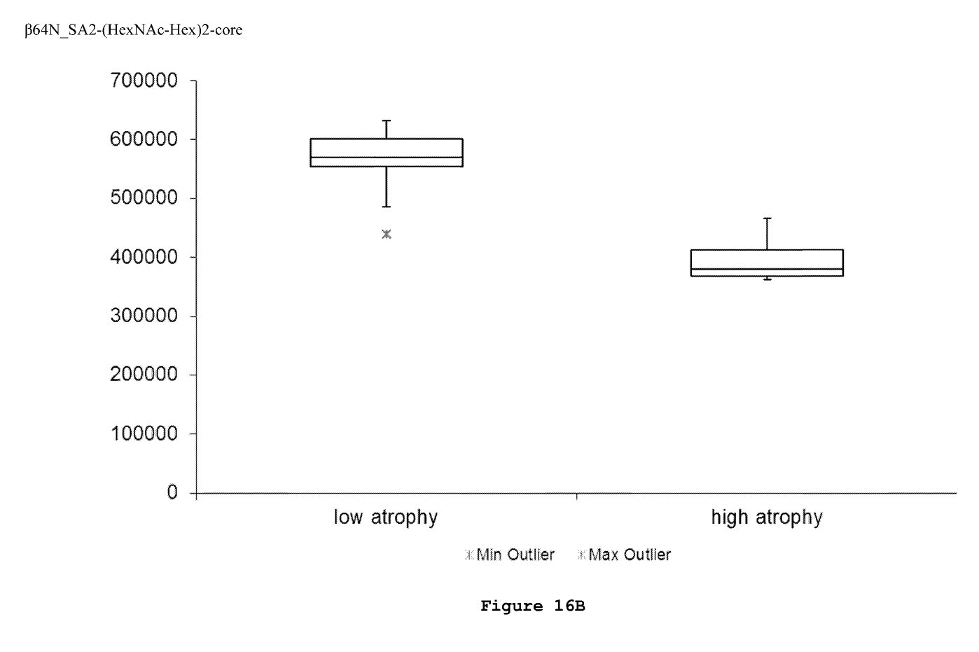

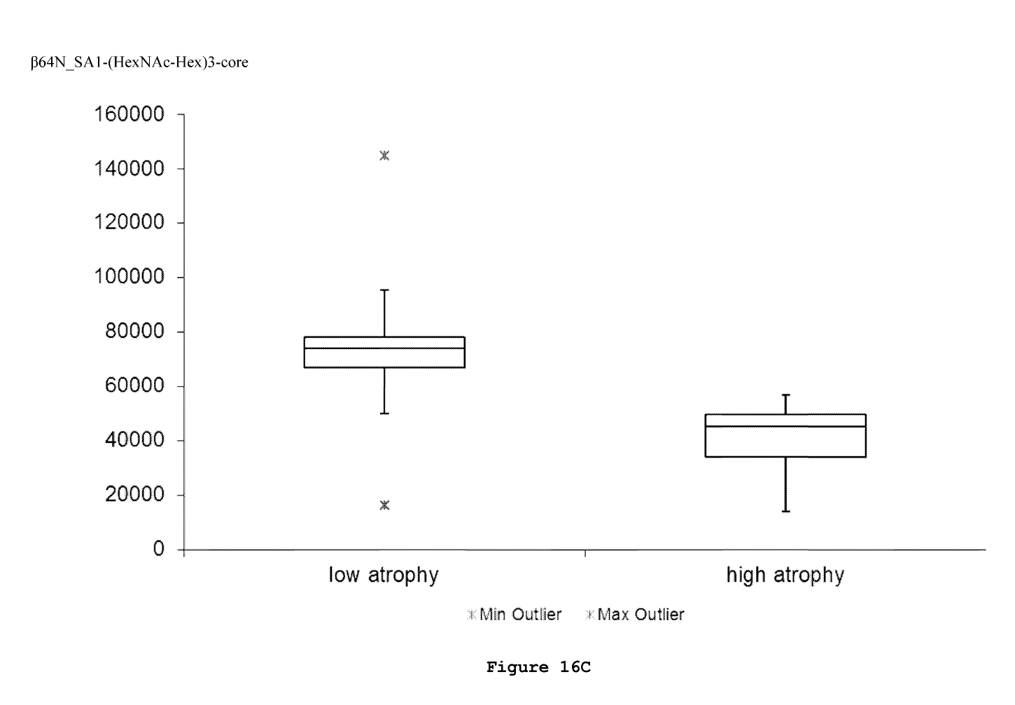

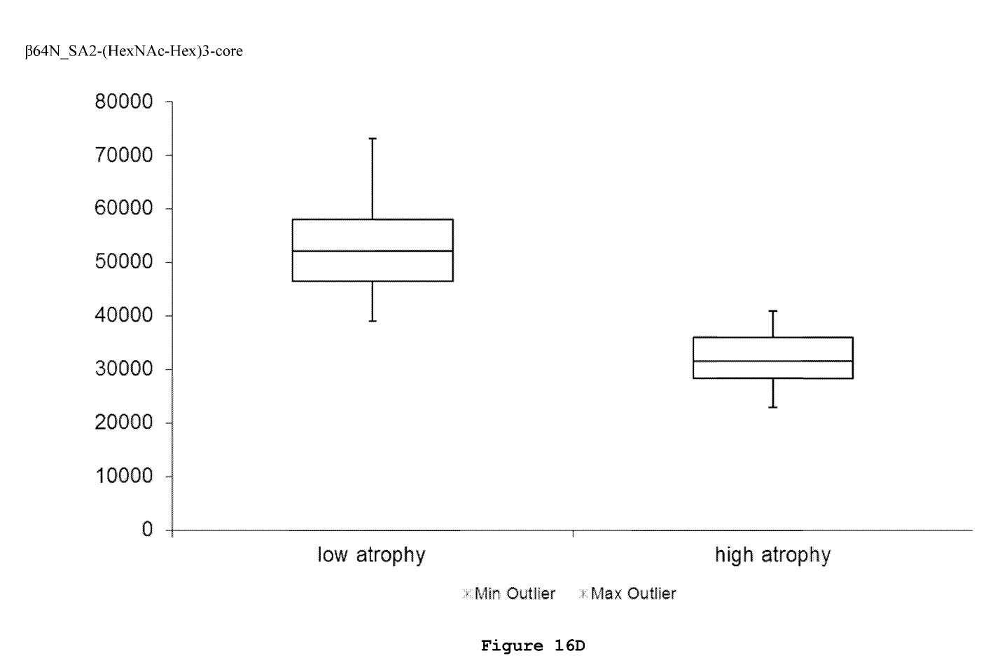

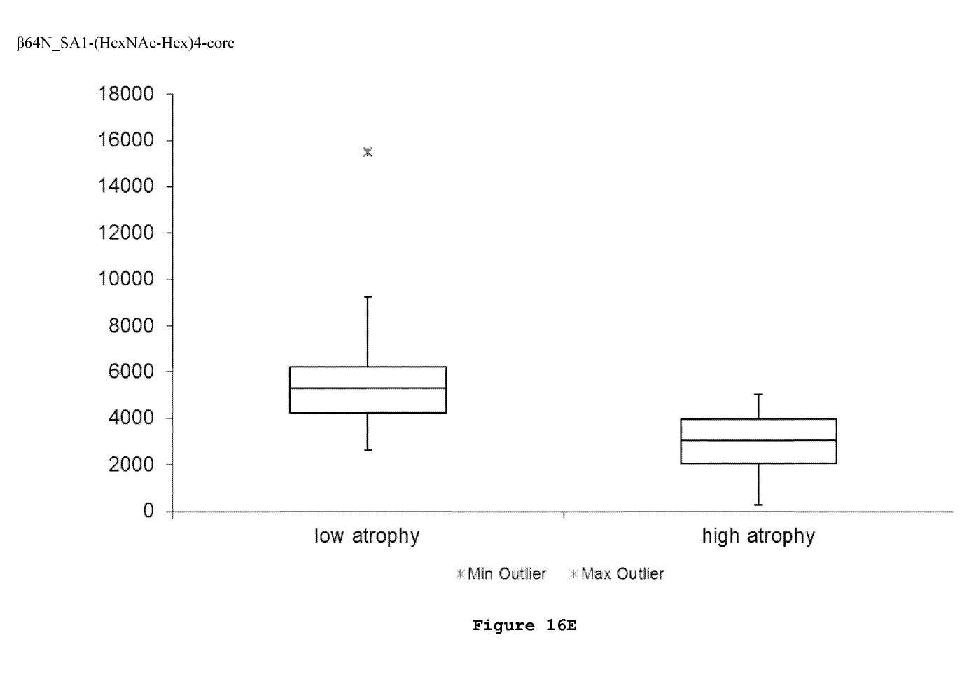

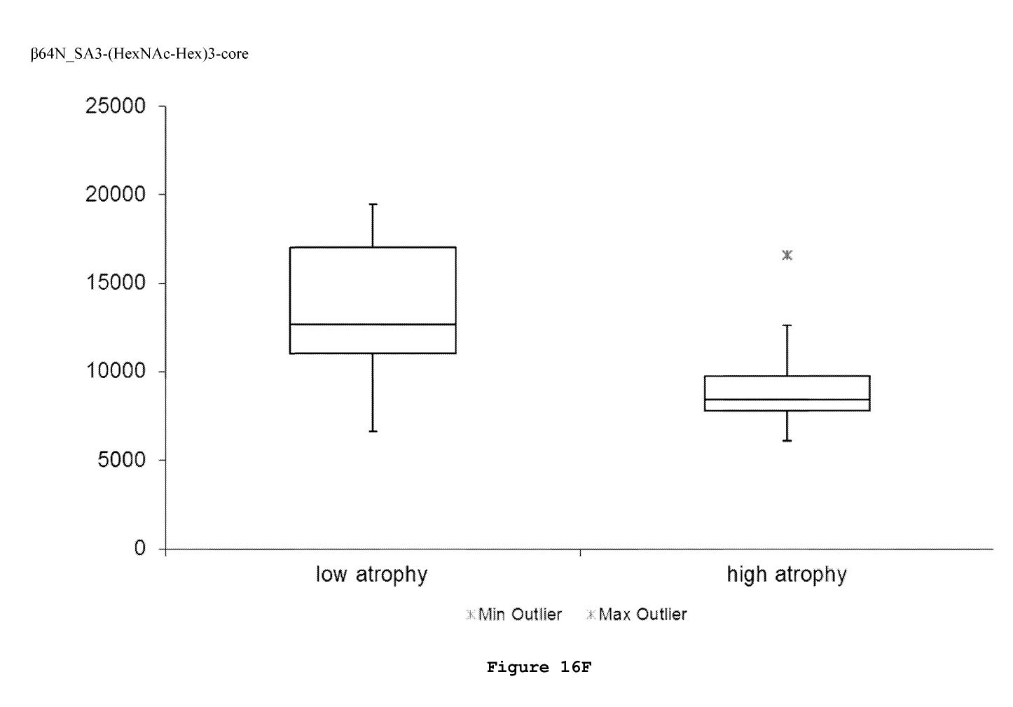

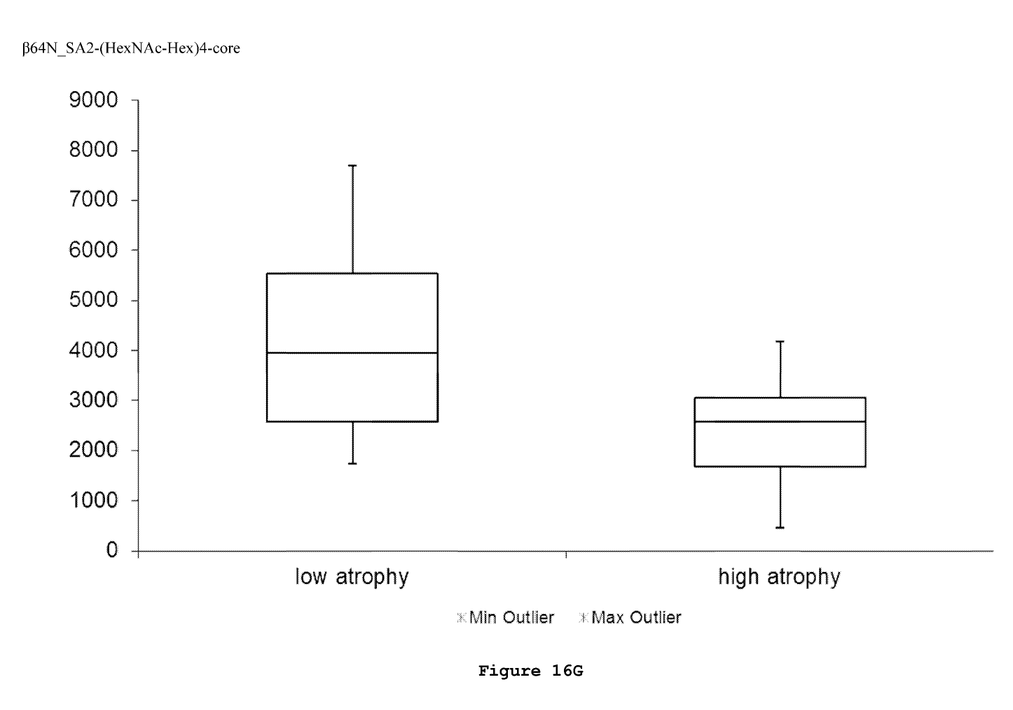

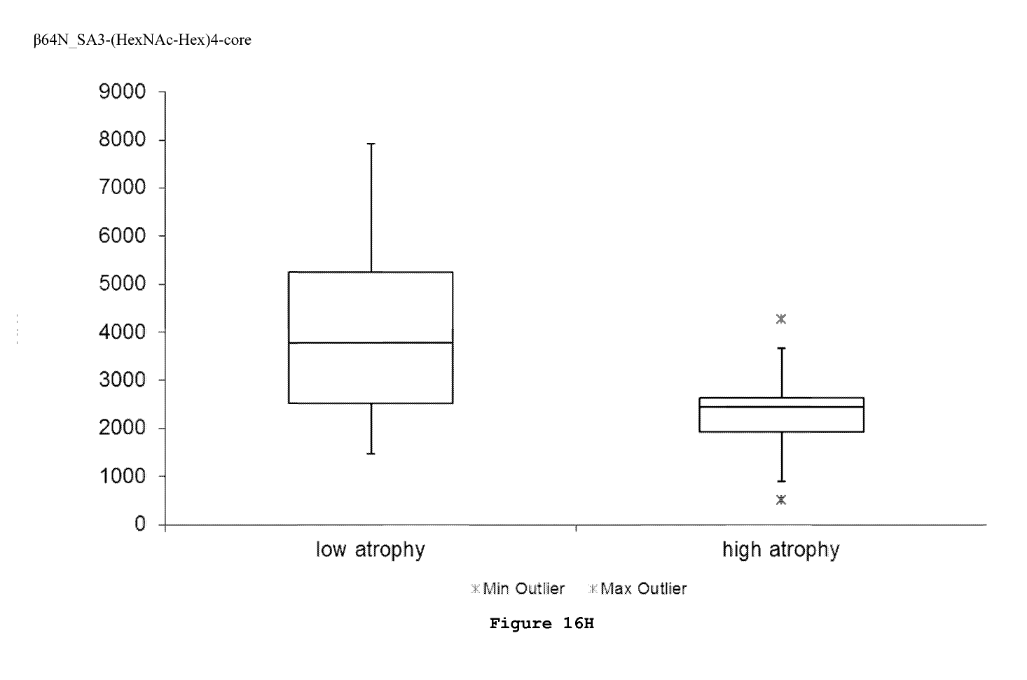

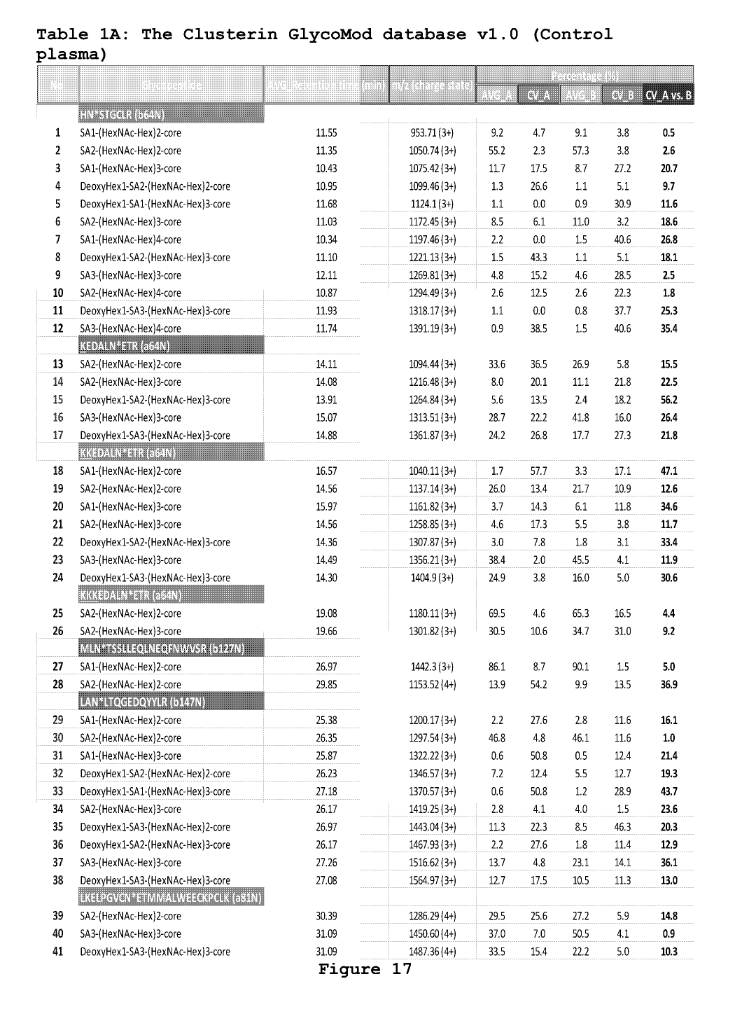

Box plots for each glycopeptide are provided in FIG. 16 (A-H, respectively).
*The p-value indicates significance of change between high and low atrophy groups.

Conclusions

A high sensitivity SRM method for eight specific N-linked glycopeptides at β64N of human clusterin can differentiate between Alzheimer's disease cases with high hippocampal atrophy and mild cognitive impairment cases with low hippocampal atrophy. This method may provide the basis for a routine clinical test to assess hippocampal atrophy based on the detection of the level of specific glycoforms in an individual patient and comparing this to levels known to represent specific levels of hippocampal atrophy. The same method may be expanded to incorporate other clusterin peptides or indeed (glyco) peptides from other plasma proteins that act as diagnostic or prognostic biomarkers of any neurodegenerative disease

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments, including application to the homologous protein biomarkers in different species are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, including patent documents, disclosed herein are incorporated by reference in their entirety for all purposes, particularly for the disclosure referenced herein.

### REFERENCES

Andreasen, N., C. Hesse, et al. (1999). “Cerebrospinal fluid beta-amyloid(1-42) in Alzheimer disease: differences between early- and late-onset Alzheimer disease and stability during the course of disease.” Arch Neurol 56(6): 673-80.
Bosman, G. J., I. G. Bartholomeus, et al. (1991). “Erythrocyte membrane characteristics indicate abnormal cellular aging in patients with Alzheimer's disease.” Neurobiol Aging 12(1): 13-8.
Butterfield, D. A., J. B. Owen (2011). “Lectin-affinity chromatography brain glycoproteomics and Alzheimer disease: insights into protein alterations consistent with the pathology and progression of this dementing disorder.”Proteomics Clin Appl. 5(1-2):50-6
Friedland, R. P. (1993). “Epidemiology, education, and the ecology of Alzheimer's disease.” Neurology 43(2): 246-9.
Ida, N., T. Hartmann, et al. (1996). “Analysis of heterogeneous A4 peptides in human cerebrospinal fluid and blood by a newly developed sensitive Western blot assay.” J Biol Chem 271(37): 22908-14.
Kanai, M., E. Matsubara, et al. (1998). “Longitudinal study of cerebrospinal fluid levels of tau, A beta1-40, and A beta1-42(43) in Alzheimer's disease: a study in Japan.” Ann Neurol 44(1): 17-26.
Kawarabayashi, T., L. H. Younkin, et al. (2001). “Age-dependent changes in brain, CSF, and plasma amyloid (beta) protein in the Tg2576 transgenic mouse model of Alzheimer's disease.” J Neurosci 21(2): 372-81.
Killick, R., E. M. Ribe, et al. (2012). “Clusterin regulates β-amyloid toxicity via Dickkopf-1-driven induction of the wnt-PCP-JNK pathway.” Mol Psychiatry. doi: 10.1038/mp.2012.163. [Epub ahead of print]
Kosaka, T., M. Imagawa, et al. (1997). “The beta APP717 Alzheimer mutation increases the percentage of plasma amyloid-beta protein ending at A beta42(43).” Neurology 48(3): 741-5.
Kuo, Y. M., T. A. Kokjohn, et al. (2000). “Elevated abeta42 in skeletal muscle of Alzheimer disease patients suggests peripheral alterations of AbetaPP metabolism.” Am J Pathol 156(3): 797-805.
Lindner, M. D., D. D. Gordon, et al. (1993). “Increased levels of truncated nerve growth factor receptor in urine of mildly demented patients with Alzheimer's disease.” Arch Neurol 50(10): 1054-60.
Nuutinen T., T. Suuronen, et al. (2009) “Clusterin: a forgotten player in Alzheimer's disease.”Brain Res Rev. 61(2):89-104.
Pirttila, T., S. Mattinen, et al. (1992). “The decrease of CD8-positive lymphocytes in Alzheimer's disease.” J Neurol Sci 107(2): 160-5.
Rocca, W. A., A. Hofman, et al. (1991). “Frequency and distribution of Alzheimer's disease in Europe: a collaborative study of 1980-1990 prevalence findings. The EURODEM-Prevalence Research Group.” Ann Neurol 30(3): 381-90.
Sato, Y., T. Endo. (2010). “Alteration of brain glycoproteins during aging.” Geriatr Gerontol Int 10 (Suppl. 1): 32-S40
Scheuner, D., C. Eckman, et al. (1996). “Secreted amyloid beta-protein similar to that in the senile plaques of Alzheimer's disease is increased in vivo by the presenilin 1 and 2 and APP mutations linked to familial Alzheimer's disease.” Nat Med 2(8): 864-70.
Ueno, I., T. Sakai, et al. (2000). “Analysis of blood plasma proteins in patients with Alzheimer's disease by two-dimensional electrophoresis, sequence homology and immunodetection.” Electrophoresis 21(9): 1832-45.
Robinson, M. D et al. (2010). “edgeR: a Bioconductor package for differential expression analysis of digital gene expression data” Bioinformatics 26: 139-140.
Paulson et al. (2013). “Differential abundance analysis for microbial marker-gene surveys” Nature Methods 10: 1200-1202.
De Livera et al. (2012). “Normalizing and Integrating Metabolomics Data” Anal. Chem. 84(24): pp. 10768-10776.
Nilselid et al. (2006). “Clusterin in cerebrospinal fluid: Analysis of carbohydrates and quantification of native and glycosylated forms” Neurochemistry International 48: 718-728.

1-44. (canceled)

45. A method for diagnosing or assessing a neurodegenerative disease or neurodegenerative dementia in a test subject, comprising:
(i) providing a protein-containing sample that has been obtained from the test subject;
(ii) determining the concentration, amount or degree of expression of at least one specific protein isoform and/or glycoform derived from a protein biomarker selected from the group consisting of: clusterin precursor; apolipoprotein A-IV precursor; apolipoprotein C-III precursor; transthyretin; galectin 7; complement C4 precursor; alpha-2-macroglobulin precursor; Ig alpha-1 chain C; histone 2B; Ig lambda chain C region; fibrinogen gamma chain precursor; complement factor H; inter-alpha-trypsin heavy chain H4 precursor; complement C3 precursor; gamma or beta actin; haptoglobin precursor; and serum albumin precursor, or a fragment thereof;
(iii) quantifying a difference between said concentration, amount or degree determined in (ii) and a reference concentration, amount or degree of the at least one specific protein isoform and/or glycoform derived from said protein biomarker from a control subject with a specific neurodegenerative disease, dementia or stage of disease, or a control subject that does not have a neurodegenerative disease or a neurodegenerative dementia; and
(iv) diagnosing or assessing the presence of or stage of a neurodegenerative disease or neurodegenerative dementia in the test subject based upon a difference in the level of the at least one specific protein isoform and/or glycoform derived from the protein biomarker in the test subject relative to the reference identified in step (iii).

46. The method according to claim 45, wherein said at least one specific protein isoform and/or glycoform is derived from clusterin precursor and comprises:
(a) a glycosylated fragment of human clusterin comprising at least 5, 6, 7, 8, 9, or 10 contiguous amino acids of the human clusterin amino acid sequence; and wherein said fragment comprises an N-linked or O-linked glycan; or
(b) a glycoform of human clusterin.

47. The method according to claim 46, wherein said glycosylated fragment of human clusterin is selected from the group consisting of:
(SEQ ID NO: 2)
HN*STGCLR;
(SEQ ID NO: 3)
KEDALN*ETR;
(SEQ ID NO: 4)
KKEDALN*ETR;
(SEQ ID NO: 5)
KKKEDALN*ETR;
(SEQ ID NO: 6)
MLN*TSSLLEQLNEQFNWVSR;
(SEQ ID NO: 7)
LAN*LTQGEDQYYLR;
(SEQ ID NO: 8)
QLEEFLN*QSSPFYFWMWGDR;
(SEQ ID NO: 9)
ELPGVCN*ETMMALWEECK;
and
(SEQ ID NO: 10)
LKELPGVCN*ETMMALWEECKPCLK;
wherein “N*” indicates the glycan attachment
residue.

48. The method according to claim 46, wherein said glycosylated fragment of human clusterin:
(a) is selected from any one of the clusterin glycopeptides set forth in Table 3A, Table 3B, Table 3C, Table 5, Table 6 and/or Table 7; or
(b) comprises a β64N-glycan selected from the group consisting of: β64N_SA1-(HexNAc-Hex)2-core; β64N_SA2-(HexNAc-Hex)2-core; β64N_SA1-(HexNAc-Hex)3-core; β64N_SA2-(HexNAc-Hex)3-core; β64N_SA1-(HexNAc-Hex)4-core; β64N_SA3-(HexNAc-Hex)3-core; β64N_SA2-(HexNAc-Hex)4-core; and β64N_SA3-(HexNAc-Hex)4-core.

49. The method according to claim 45, wherein said at least one specific protein glycoform is a tetra-antennary glycoform of the protein biomarker.

50. The method according to claim 45, wherein said concentration, amount or degree of expression of the at least one specific protein isoform and/or glycoform is determined:
(i) relative to at least one other isoform and/or glycoform of the same protein or relative to the total of all isoforms and/or glycoforms of the same protein;
(ii) relative to a reference protein other than one of said protein biomarkers; or
(iii) using a sum-scaling method in which one or more raw values of said concentration, amount or degree of expression are normalised to give a normalised sum-scaled measurement; wherein the concentration, amount or degree of expression of a tetra-antennary glycoform of the protein biomarker is determined relative to one or more lower antennary glycoforms of the same protein or relative to the total of all glycoforms of the same protein.

51. The method according to claim 50, wherein the method comprises determining the proportion of tetra-antennary glycoforms of the protein biomarker relative to the total of all glycoforms of the same protein; wherein the method comprises quantifying tetra-antennary glycoforms of a human clusterin glycoprotein fragment comprising the sequence HN*STGCLR (SEQ ID NO: 2) as a proportion of the total of all glycoforms of the same glycoprotein fragment.

52. The method according to claim 51, wherein a lower relative level of tetra-antennary glycoforms in the sample from the test subject compared with the relative level of tetra-antennary glycoforms in the reference from the control subject indicates that the test subject is predicted to have a neurodegenerative disease or dementia or to have a more advanced stage of neurodegenerative disease or dementia; wherein said more advanced stage of neurodegenerative disease or dementia comprises a relatively higher level of hippocampal atrophy.

53. The method according to claim 45, wherein said neurodegenerative disease or neurodegenerative dementia is selected from the group consisting of Alzheimer's disease (AD), Mild Cognitive Impairment (MCI), vascular dementia, dementia with Lewy bodies, frontotemporal dementia alone or as a mixed dementia with AD, Parkinson's disease, and Huntington's disease.

54. The method according to claim 45, wherein the method comprises determining the concentration, amount or degree of expression of at least one specific protein isoform and/or glycoform of each of at least two, three, four or five of said biomarker proteins.

55. The method according to claim 45, wherein the protein-containing sample is selected from the group consisting of: blood plasma, blood cells, serum, saliva, urine, cerebro-spinal fluid (CSF), cell scraping, and a tissue biopsy.

56. The method according to claim 45, wherein the protein isoforms and/or glycoforms are glycoforms and are measured using:
(a) gel electrophoresis; or
(b) LC-MS/MS; and wherein the relative amount of each glycoform is calculated by comparison to an equivalent heavy-isotope labelled reference glycoform using Selected Reaction Monitoring (SRM) mass spectrometry.

57. The method according to claim 45, wherein the protein isoforms and/or glycoforms are glycoforms and are:
(a) measured using sum scaled Selected Reaction Monitoring (SRM) mass spectrometry;
(b) not labelled; or
(c) measured using gel electrophoresis followed by Selected Reaction Monitoring (SRM) mass spectrometry.

58. The method according to claim 56, wherein the heavy-isotope labelled reference glycoform is:
(a) a synthetic glycopeptide with one or more heavy isotopes of H, C, N or O are substituted within the peptide or sugar components of said glycoform; or
(b) an enriched, naturally occurring glycoform that has been labelled with an isotopic mass tag wherein said isotopic mass tag with one or more heavy isotopes of H, C, N or O and wherein such mass tag is able to react with the peptide or sugar components of said glycoform.

59. The method according to claim 56, wherein the relative amount of each glycoform is calculated by comparison to an equivalent glycoform labelled with an isobaric mass tag wherein:
(i) each sample of tissue or body fluid taken from the test subject is labelled with one member of an isobaric mass tag set to create a labelled analytical sample;
(ii) a standard reference panel of enriched glycoforms is separated into between two and six aliquots and each aliquot is labelled separately with additional members of the same isobaric mass tag set as the labelled analytical sample and each independently labelled aliquot of the reference panel is mixed in a predefined ratio to create a clinically relevant concentration curve as a standard reference mixture;
(iii) an equal volume of the labelled analytical sample and the standard reference mixture are mixed together to form a MS calibrator sample; and
(iv) the MS calibrator sample prepared in step (iii) is analysed by mass spectrometry;
wherein the isobaric mass tag set is a Tandem Mass Tag set.

60. The method according to claim 45, wherein the at least one specific protein isoform and/or glycoform is measured by an immunological assay, wherein the immunological assay comprises Western blotting or ELISA.

61. A method for stratifying a plurality of test subjects according to their stage or severity of neurodegenerative disease or dementia, comprising:
carrying out the method according to claim 45 on at least one test sample from each of the plurality of test subjects; and
stratifying the test subjects into more or less advanced stage neurodegenerative disease or dementia or into more or less severe neurodegenerative disease or dementia based on the level of the at least one specific protein isoform and/or glycoform of the protein biomarker in each of the test subjects.

62. The method for stratifying a plurality of test subjects according to claim 61, wherein the test subjects are stratified according to their predicted degree of hippocampal atrophy.

63. A method of determining the efficacy of a treatment of a neurodegenerative disease or neurodegenerative dementia in a mammalian subject, comprising:
determining the level of one or more isoforms and/or glycoforms of at least one protein biomarker by the method according to claim 45 before treatment of the subject and at least one additional time during or following treatment of the subject;
wherein successful treatment is demonstrated by the level of the said one or more isoforms and/or glycoforms remaining stable or reverting to more normal levels; and
wherein the subject is a human, a mouse, or a rat.

64. A method of treating a neurodegenerative disease or dementia in a subject diagnosed with said neurodegenerative disease or dementia by the administration of a therapeutic amount of an inhibitor of β-N-acetyl-glucosaminidase, wherein the subject has been diagnosed by the method according to claim 45.
